SEQUENCE LISTING (103 sequences)

SEQ ID NO: 1 - protein, 20 aa, synthetic construct
SEFAYGSFVRTVSLPVGADE

SEQ ID NO: 2 - protein, 20 aa, synthetic construct
SEFAYGSFMRSVTLPPGADE

SEQ ID NO: 3 - protein, 20 aa, synthetic construct
SEFSYGSFVRTVSLPGGADE

SEQ ID NO: 4 - protein, 20 aa, synthetic construct
SEFSYGSFVRTVSLPAGADE

SEQ ID NO: 5 - protein, 20 aa, synthetic construct
SEFSYGSFVRTVTLPTDADE

SEQ ID NO: 6 - protein, 20 aa, synthetic construct
SEFSYGSFARTVSLPAGANE

SEQ ID NO: 7 - protein, 15 aa, synthetic construct
WRMYFSHRHAHLRSP

SEQ ID NO: 8 - protein, 15 aa, synthetic construct
WHWRHRIPLQLAAGR

SEQ ID NO: 9 - protein, 12 aa, synthetic construct
HSFKWLDSPRLR

SEQ ID NO: 10 - protein, 12 aa, synthetic construct
ISLTEWSMWYRH

SEQ ID NO: 11 - protein, 24 aa, synthetic construct
ISLTEWSMWYRHHSFKWLDSPRLR

SEQ ID NO: 12 - protein, 32 aa, synthetic construct
SEFAYGSFVRTVSLPVGADEHSFKWLDSPRLR

SEQ ID NO: 13 - protein, 13 aa, synthetic construct
AEKAGGGGGAEKA

SEQ ID NO: 14 - protein, 19 aa, synthetic construct
AEKAEKAGGGGGAEKAEKA

SEQ ID NO: 15 - protein, 27 aa, synthetic construct
AEKAGGGGGAEKAQYIKANSKFIGITE

SEQ ID NO: 16 - protein, 4 aa, synthetic construct
AEKA

SEQ ID NO: 17 - protein, 9 aa, synthetic construct
AEKAGGGGG

SEQ ID NO: 18 - protein, 19 aa, synthetic construct
DWSGYSGSFVQHPELTGLD

SEQ ID NO: 19 - protein, 16 aa, synthetic construct
KSCINRCFYVELIRGR

SEQ ID NO: 20 - protein, 15 aa, synthetic construct
FVIREPFISCSHLEC

SEQ ID NO: 21 - protein, 6 aa, synthetic construct
GNFIAP

SEQ ID NO: 22 - protein, 6 aa, synthetic construct
GNLIAP

SEQ ID NO: 23 - protein, 7 aa, synthetic construct
ETPIRNE

SEQ ID NO: 24 - protein, 8 aa, synthetic construct
HYEECSCY

SEQ ID NO: 25 - protein, 7 aa, synthetic construct
IWGIHHP

SEQ ID NO: 26 - protein, 7 aa, synthetic construct
IWGVHHP

SEQ ID NO: 27 - protein, 10 aa, synthetic construct
LLTEVETPIR

SEQ ID NO: 28 - protein, 27 aa, synthetic construct
LLTEVETPIRNESLLTEVETPIRNEWG

SEQ ID NO: 29 - protein, 13 aa, synthetic construct
LLTEVETPIRNEW

SEQ ID NO: 30 - protein, 14 aa, synthetic construct
LLTEVETPIRNEWG

SEQ ID NO: 31 - protein, 11 aa, synthetic construct
LTEVETPIRNE

SEQ ID NO: 32 - protein, 12 aa, synthetic construct
LTEVETPIRNEW

SEQ ID NO: 33 - protein, 13 aa, synthetic construct
LTEVETPIRNEWG

SEQ ID NO: 34 - protein, 9 aa, synthetic construct
MSLLTEVET

SEQ ID NO: 35 - protein, 10 aa, synthetic construct
MSLLTEVETP

SEQ ID NO: 36 - protein, 11 aa, synthetic construct
MSLLTEVETPI

SEQ ID NO: 37 - protein, 12 aa, synthetic construct
MSLLTEVETPIR

SEQ ID NO: 38 - protein, 13 aa, synthetic construct
MSLLTEVETPIRN

SEQ ID NO: 39 - protein, 14 aa, synthetic construct
MSLLTEVETPIRNE

SEQ ID NO: 40 - protein, 20 aa, synthetic construct
MSLLTEVETPIRNETPIRNE

SEQ ID NO: 41 - protein, 15 aa, synthetic construct
MSLLTEVETPIRNEW

SEQ ID NO: 42 - protein, 16 aa, synthetic construct
MSLLTEVETPIRNEWG

SEQ ID NO: 43 - protein, 24 aa, synthetic construct
MSLLTEVETPIRNEWGCRCNDSSD

SEQ ID NO: 44 - protein, 8 aa, synthetic construct
SLLTEVET

SEQ ID NO: 45 - protein, 13 aa, synthetic construct
SLLTEVETPIRNE

SEQ ID NO: 46 - protein, 14 aa, synthetic construct
SLLTEVETPIRNEW

SEQ ID NO: 47 - protein, 15 aa, synthetic construct
SLLTEVETPIRNEWG

SEQ ID NO: 48 - protein, 21 aa, synthetic construct
SLLTEVETPIRNEWGTPIRNE

SEQ ID NO: 49 - protein, 27 aa, synthetic construct
SLLTEVETPIRNEWGTPIRNETPIRNE

SEQ ID NO: 50 - protein, 33 aa, synthetic construct
SLLTEVETPIRNEWGTPIRNETPIRNETPIRNE

SEQ ID NO: 51 - protein, 39 aa, synthetic construct
SLLTEVETPIRNEWGLLTEVETPIRQYIKANSKFIGITE

SEQ ID NO: 52 - protein, 10 aa, synthetic construct
TEVETPIRNE

SEQ ID NO: 53 - protein, 6 aa, synthetic construct
WGIHHP

SEQ ID NO: 54 - protein, 6 aa, synthetic construct
WGVHHP

SEQ ID NO: 55 - protein, 7 aa, synthetic construct
YPKCDRA

SEQ ID NO: 56 - protein, 9 aa, synthetic construct
WDYPKCDRA

SEQ ID NO: 57 - protein, 23 aa, synthetic construct
SLDQINVTFLDLEYEMKKLEESY

SEQ ID NO: 58 - protein, 14 aa, synthetic construct
KWPWYIWLGFIAGL

SEQ ID NO: 59 - protein, 8 aa, synthetic construct
ENQKLIAN

SEQ ID NO: 60 - protein, 11 aa, synthetic construct
DLWSYNAELLV

SEQ ID NO: 61 - protein, 11 aa, synthetic construct
DIWTYNAELLV

SEQ ID NO: 62 - protein, 23 aa, synthetic construct
SLDQINVTFLDLEYEMKKLEESY

SEQ ID NO: 63 - protein, 32 aa, synthetic construct
WDYPKCDRASLDQINVTFLDLEYEMKKLEESY

SEQ ID NO: 64 - protein, 8 aa, synthetic construct
ARDLICAQ

SEQ ID NO: 65 - protein, 21 aa, synthetic construct
YFPLQSYGFQPTNGVGYQPYR

SEQ ID NO: 66 - protein, 15 aa, synthetic construct
WRMYFSHRHAHLRSP

SEQ ID NO: 67 - protein, 15 aa, synthetic construct
WHWRHRIPLQLAAGR

SEQ ID NO: 68 - protein, 7 aa, synthetic construct
QEINSSY

SEQ ID NO: 69 - protein, 7 aa, synthetic construct
APPHALS

SEQ ID NO: 70 - protein, 7 aa, synthetic construct
VVPTPPY

SEQ ID NO: 71 - protein, 7 aa, synthetic construct
SMPNPMV

SEQ ID NO: 72 - protein, 7 aa, synthetic construct
GLQQVLL

SEQ ID NO: 73 - protein, 7 aa, synthetic construct
ELAPDSP

SEQ ID NO: 74 - protein, 12 aa, synthetic construct
  VARIANT 8: note = Any amino acid
STLNYMYXAHPF

SEQ ID NO: 75 - protein, 12 aa, synthetic construct
ISLSNIVDSQTP

SEQ ID NO: 76 - protein, 12 aa, synthetic construct
GFSVITGAAMFE

SEQ ID NO: 77 - protein, 13 aa, synthetic construct
RKSIHLGPGRAFY

SEQ ID NO: 78 - protein, 13 aa, synthetic construct
KKGIAIGPGRTLY

SEQ ID NO: 79 - protein, 13 aa, synthetic construct
RKSIRIGPGQAFY

SEQ ID NO: 80 - protein, 13 aa, synthetic construct
RKRIRVGPGQTVY

SEQ ID NO: 81 - protein, 19 aa, synthetic construct
NPDPNANPNVDPNANGGGC

SEQ ID NO: 82 - protein, 7 aa, synthetic construct
GNLFIAP

SEQ ID NO: 83 - protein, 7 aa, synthetic construct
GNLIFAP

SEQ ID NO: 84 - protein, 8 aa, synthetic construct
IWGVIHHP

SEQ ID NO: 85 - protein, 8 aa, synthetic construct
IWGIVHHP

SEQ ID NO: 86 - protein, 7 aa, synthetic construct
WGVIHHP

SEQ ID NO: 87 - protein, 7 aa, synthetic construct
WGIVHHP

SEQ ID NO: 88 - protein, 9 aa, synthetic construct
YIWGVIHHP

SEQ ID NO: 89 - protein, 9 aa, synthetic construct
YIWGIVHHP

SEQ ID NO: 90 - protein, 22 aa, synthetic construct
GNLFIAPWGVIHHPHYEECSCY

SEQ ID NO: 91 - protein, 22 aa, synthetic construct
WGVIHHPGNLFIAPHYEECSCY

SEQ ID NO: 92 - protein, 42 aa, synthetic construct
SEFAYGSFVRTVSLPVGADEGNLFIAPWGVIHHPHYEECSCY

SEQ ID NO: 93 - protein, 42 aa, synthetic construct
GNLFIAPWGVIHHPHYEECSCYSEFAYGSFVRTVSLPVGADE

SEQ ID NO: 94 - protein, 28 aa, synthetic construct
HYEECSCYSEFAYGSFVRTVSLPVGADE

SEQ ID NO: 95 - protein, 25 aa, synthetic construct
SLLTEVETPIRNEWGLLTEVETPIR

SEQ ID NO: 96 - protein, 25 aa, synthetic construct
HYEECSCYWDYPKCDRAVETPIRNE

SEQ ID NO: 97 - protein, 26 aa, synthetic construct
ENQKLIANTEVETPIRNEHYEECSCY

SEQ ID NO: 98 - protein, 20 aa, synthetic construct
HYEECSCYHSFKWLDSPRLR

SEQ ID NO: 99 - protein, 32 aa, synthetic construct
NPDPNANPNVDPNANGGGCRKSIHLGPGRAFY

SEQ ID NO: 100 - protein, 36 aa, synthetic construct
  VARIANT 8: note = Any amino acid
STLNYMYXAHPFWRMYFSHRHAHLRSPGGGGGAEKA

SEQ ID NO: 101 - protein, 21 aa, synthetic construct
NMMRFTSQPPNNGGGGGAEKA

SEQ ID NO: 102 - protein, 44 aa, synthetic construct
NPDPNANPNVDPNANGGGCHSFKWLDSPRLRRKSIRIGPGQAFY

SEQ ID NO: 103 - protein, 41 aa, synthetic construct
AEKAGGGGGHSFKWLDSPRLRSEFAYGSFVRTVSLPVGADE

(19) United States
(12) Patent Application Publication
(10) Pub. No.: US 2025/0325654 A1
(43) Pub. Date: Oct. 23, 2025

(54) Vaccines and Antibodies for the Treatment and Prevention of Microbial Infections

(71) Applicant: Longhorn Vaccines and Diagnostics, LLC, Bethesda, MD (US)

(72) Inventors: Gerald W. Fischer, Bethesda, MD (US); Clara J. Sei, Germantown, MD (US); Jeffrey D. Fischer, Washington, DC (US); Richard F. Schuman, Silver Spring, MD (US)

(73) Assignee: Longhorn Vaccines and Diagnostics, LLC, BETHESDA, MD (US)

(21) Appl. No.: 19/182,300

(22) Filed: Apr. 17, 2025

Related U.S. Application Data
(60) Provisional application No. 63/635,703, filed on Apr. 18, 2024.

(51) Int. Cl. A61K 39/215; A61K 39/00; A61K 39/04; A61K 39/145; A61K 39/39; A61P 31/04; A61P 31/06; A61P 31/16

(52) U.S. Cl. CPC A61K 39/215; A61K 39/04; A61K 39/145; A61K 39/39; A61P 31/04; A61P 31/06; A61P 31/16; A61K 2039/55555; A61K 2039/55566; A61K 2039/55572; A61K 2039/55577

(57) ABSTRACT

The invention relates to low dose compositions and peptides or peptide sequences that induce an immune response in an animal or a mammal that is protective against infection by one or more pathogens, and the antibodies generated. In addition, the invention relates to immunogenic composition and vaccines comprising compositions and peptide sequences or antibodies, and to methods for treating and preventing an infection in animals and mammals such as humans.

### REFERENCE TO RELATED APPLICATIONS

This application claims priority to U.S. Provisional Application No. 63/662,027 filed Jun. 20, 2024, and U.S. Provisional Application No. 63/635,703 filed Apr. 18, 2024, the entirety of each of which is specifically incorporated by reference.

### SEQUENCE LISTING

The instant application contains a Sequence Listing submitted electronically in XML format and is hereby incorporated by reference in its entirety. Said XML copy, created on Apr. 17, 2025, is named 3022_060_US_SL.xml and is 91,399 bytes in size.

### BACKGROUND

### 1 Field of the Invention

The present invention is directed to immunogenic antigens and vaccines composed of a plurality of epitopes of one or more pathogens, and to tools and methods for generating an immune response. In particular, the invention is directed to compositions comprising microbial specific peptides and/or nucleic acid sequences as vaccines for the treatment and prevention of microbial diseases.

### 2 Description of the Background

Respiratory viruses cause severe infections in children and adults. For example, influenza viruses are etiologic agents for a contagious respiratory illness (commonly referred to as the flu) that primarily affects humans and other vertebrates. Influenza is highly infectious and an acute respiratory disease that has plagued the human race since ancient times. Infection is characterized by recurrent annual epidemics and periodic major worldwide pandemics. Influenza virus infection can cause mild to severe illness and can even lead to death. Every year in the United States, 5 to 20 percent of the population, on average, contracts the flu with more than 200,000 hospitalizations from complications and over 36,000 deaths. Because of the high disease-related morbidity and mortality, direct and indirect social economic impacts of influenza are enormous. Four pandemics occurred in the last century, together causing tens of millions of deaths worldwide.

The CDC and the leading authorities on disease prevention in the world recommend the single best way of preventing a viral respiratory infection in humans is through regular vaccinations. Vaccines for many respiratory viruses if available, would be useful to prevent much human disease and suffering. Conventional influenza are not ideal because the vaccines typically target the immunodominant protein HA. These vaccines have not been universally protective or 100 percent effective at preventing the disease. Antigenic shift prevents flu vaccines from being universally protective or from maintaining effectiveness over many years. The ineffectiveness of conventional vaccines may also be due, in part, to antigenic drift and the resulting variation within antigenic portions of the HA protein most commonly recognized by the immune system. As a result, many humans may find themselves susceptible to the flu virus without an effective method of treatment available since influenza is constantly improving its resistance to current treatments. This scenario is particularly concerning with respect to the H5N1 virus, which is highly virulent but for which there is currently no widely available commercial vaccine to immunize susceptible human populations.

Currently, flu vaccines are reformulated each year due to the yearly emergence of new strains, and generally induce limited immunity. In addition, to achieve a protective immune response, some vaccines are administered with high doses of antigen. This is particularly true for H5N1 vaccines. In addition, influenza vaccines, including H5N1 vaccines, typically present epitopes in the same order as the epitopes are found in nature, generally presenting as whole-viral proteins; consequently, relatively large amounts of protein are required to make an effective vaccine. As a result, each administration includes an increased cost associated with the dose amount, and there is increased difficulty in manufacturing enough doses to vaccinate the general public. Further, the use of larger proteins elevates the risk of undesirable immune responses in the recipient host.

Approximately one third of the world population is infected with Mycobacterium tuberculosis (MTB). Mycobacteria belong to the diverse family of Actinobacteria. The main components of the mycobacterial cell wall and many other microorganisms are the peptidoglycan (PGN) layer, mycolic acid (MA), arabinogalactan (AG), lipomannans (LPM), mycolic acids (MA), and lipid containing molecules such as lipopolysaccharide (LPS), lipoteichoic acids (LTA), and lipoarabinomannan (LAM). The mycobacterial cell wall resembles both the Gram-positive and Gram-negative cell envelope by having a PGN layer nearly as thick as the former and an outer, waxy layer mimicking the outer membrane of the latter. Current treatment includes a long course of antibiotics and often requires quarantining of the patient. Resistance is common and an ever-increasing problem, as is the ability to maintain the quarantine of infected patients. Present vaccines include BCG which is prepared from a strain of attenuated (virulence-reduced) live bovine tuberculosis bacillus, Mycobacterium bovis, and live non-MTB organisms. BCG carries substantial associated risks, especially in immune compromised individuals, and has proved to be only modestly effective and for limited periods. It is generally believed that a humoral response to infection by MTB is ineffective and optimal control of infection must involve activation of T cells and macrophages. As MTB is a human pathogen, research on MTB is often conducted using Mycobacterium smegmatis, which is considered sufficiently similar, but is not pathogenic to humans. In addition, HIV and malaria continue to infect many people causing suffering and death across the globe. Effective vaccines are presently unavailable and greatly needed.

Within an immune response, T cells are important tools of the immune system and a major source of the cascade of cytokines that occurs following an immune response. Two of the principal forms of T cells are identified by the presence of the cell surface molecules CD4 and CD8. T cells that express CD4 are generally referred to as helper T cells. T helper cells include the subsets Th1 and Th2, and the cytokines they produce are known as Th1-type cytokines and Th2-type cytokines, both sets of which are of critical importance in developing an immune response. The Th1-type cytokines produce a pro-inflammatory response stimulating the opsonization of intracellular parasites, basically the humoral immune response. Interferon gamma is one of the principal Th1 cytokines. The Th2-type cytokines include interleukins 4, 5, 10 and 13, which are closely associated with the promotion of a cellular immune response. Against an infection, a balanced Th1 and Th2 response is most desired.

Protective anti-microbial vaccines are greatly needed that provide protection against or treatment of infection by multiple different microbes including different serotypes, species, and genus of virus, bacteria, fungus, and/or parasites. It is further needed that such vaccines be efficiently and economically produced.

### SUMMARY OF THE INVENTION

The present invention provides new and useful compositions, as well as tools and methods directed to immunogenic compositions, vaccines and antibodies against one or more pathogens for treating and/or preventing infections in mammals such as humans, a viral, bacterial, fungal, or parasitic infection and enhancing the immune system of a patient.

One embodiment of the invention is directed to peptides containing one or more and preferably multiple viral, bacterial, fungal, and/or parasitic epitopes. mimotopes and/or composite epitopes. Peptides of the invention may comprise multiple viral epitopes, bacterial epitopes, and/or parasitic epitopes, or preferably combination of different epitopes of different microbes. The peptides may be part of an immunogenic composition which may optionally contain an adjuvant such as, for example, Freund's, a liposome, saponin, lipid A, squalene, and derivatives and combinations thereof. Preferred adjuvants include, for example, AS01 (Adjuvant System 01) which is a liposome-based adjuvant which comprises QS-21 (a saponin fraction extracted from Quillaja saponaria Molina), and 3-O-desacyl-4′-monophosphoryl lipid A (MPL; a non-toxic derivative of the lipopolysaccharide from Salmonella minnesota) and on occasion a ligand such as a toll-like receptor (e.g., TLR4), AS01b which is a component of the adjuvant Shingrix, ALF (Army Liposome Formulation) which comprises liposomes containing saturated phospholipids, cholesterol, and/or monophosphoryl lipid A (MPLA) as an immunostimulant. ALF is safe to use in humans (e.g., has no harmful clinical effects), and increases potency of the vaccine component. AS01 is included in the malaria vaccine RTS, S (MOSQUIRIX®). ALF modifications and derivatives include, for example, ALF adsorbed to aluminum hydroxide (ALFA), ALF containing QS21 saponin (ALFQ), and ALFQ adsorbed to aluminum hydroxide (ALFQA). A preferred adjuvant formulation comprises Freund's adjuvant, a liposome, saponin, lipid A, squalene, unilamellar liposomes having a liposome bilayer that comprises at least one phosphatidylcholine (PC) and/or phosphatidylglycerol (PG), as phospholipids, which may be dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), and/or distearyl phosphatidylglycerol (DSPG), a cholesterol, a monophosphoryl lipid A (MPLA), and a saponin. Preferably, the immunogenic composition is a vaccine that treats or prevents a viral, bacterial or parasitic infection in humans, mammals and other animals including but not limited to porcine and avian species.

Another embodiment of the invention comprises a peptide containing one or more microbial epitopes, mimotopes, and/or composite epitopes such as, for example, as described herein, and one or more T cell stimulating epitopes. The T cell stimulating epitope is obtained or derived from tetanus toxin, tetanus toxin heavy chain proteins, diphtheria toxoid, cross reactive material (CRM and CRM₁₉₇), synthesized or recombinantly produced CRM, tetanus toxoid, Pseudomonas exoprotein A, Pseudomonas aeruginosa toxoid, Bordetella pertussis toxoid, Clostridium perfringens toxoid, Escherichia coli heat-labile toxin B subunit, Neisseria meningitidis outer membrane complex, Hemophilus influenzae protein D, Flagellin Fli C, Horseshoe crab Haemocyanin, and/or a fragment, derivative, or modification thereof. Preferably the T cell stimulating epitope is at the N-terminus or the C-terminus of the peptide. Peptides of the invention may comprise multiple microbial epitopes and/or multiple T cell stimulating epitopes. Peptides of the disclosure may be part of an immunogenic composition which may optionally contain an adjuvant such as, for example, Freund's, ALFQ, ALFQA, ALFA, AS01, AS01b, a liposome, saponin, lipid A, squalene, oil in water emulsion and derivatives and combinations thereof. Preferably, the immunogenic composition is a vaccine that treats and/or prevents a viral, bacterial or parasitic infection in humans, mammals and other animals.

Another embodiment of the invention comprises a peptide containing one or more microbial epitopes. Mimotopes, and/or composite epitopes, and optionally one or more T cell stimulating epitopes and/or one or more adjuvants, all as described herein, which are prepared in dosage form. Doses may be maintained in individual glass or plastic containers such as, for example, vials or syringes for individual administration to a subject. Alternatively, doses may be combined in a larger container for administration to large numbers of subjects such as, for example, collections of mammals (e.g., equine, bovine, porcine, caprine, ovine) or animals such as birds (e.g. fowl, chickens, turkeys) and for distribution to wildlife.

Other embodiments and advantages of the invention are set forth in part in the description, which follows, and in part, may be obvious from this description, or may be learned from the practice of the invention.

### DESCRIPTION OF THE FIGURES

FIG. 1A Chart of antigen components, immunizations, hybridoma target proteins, and mAb binding.

FIG. 1B Schematic of process for the production of hybridoma cells.

FIG. 2A Monoclonal binding activity of LD9 (IgG1) against influenza antigens A/H1N1, A/H3N2, A/H5N1, and B/Victoria strain.

FIG. 2B Monoclonal binding activity of NB5 (IgG2a) against influenza antigens A/H1N1, A/H3N2, A/H5N1, and B/Victoria strain.

FIG. 2C Monoclonal binding activity of GA4 (IgG1) against influenza antigens A/H1N1, A/H3N2, A/H5N1, and B/Victoria strain.

FIG. 2D Monoclonal binding activity of CG6 (IgG3) against influenza antigens A/H1N1, A/H3N2, A/H5N1, and B/Victoria strain.

FIG. 3 Neutralizing activity of LD9, NB5, GA4, and CG6 against Group 1 and Group 2 influenza A virus.

FIG. 4A Binding activity of LD7 against 105 live MTB.

FIG. 4B Binding activity of LD7 against 105 ethanol-killed MTB.

FIG. 4C Binding activity of LD7 against 106 live MTB.

FIG. 4D Binding activity of LD7 against 106 ethanol-killed MTB.

FIG. 5A Binding activity of MD11 against 105 live MTB.

FIG. 5B Binding activity of MD11 against 105 ethanol-killed MTB.

FIG. 6 Comparison of binding activities of LD7 and MD 11 against 105 live MTB.

FIG. 7 Composition of LHNVD-105 composite peptide vaccine.

FIG. 8 Study design of composite peptide vaccine.

FIG. 9 Study design of vaccination schedule,

FIG. 10 Binding activity of pig antisera to LHNVD-105.

FIG. 11A Binding activity of pig antisera at day 21 to whole virus (H1N1).

FIG. 11B Binding activity of pig antisera at day 56 to whole virus (H1N1).

FIG. 11C Binding activity of pig antisera at day 21 to whole virus (H3N2).

FIG. 11D Binding activity of pig antisera at day 56 to whole virus (H3N2).

FIG. 12A Neutralizing titer of doses of pig antisera to FluA virus (H1N1).

FIG. 12B Neutralizing titer of doses of pig antisera to FluA virus (H3N2).

FIG. 13A Hemagglutinin inhibition of pig antisera to FluA virus (H1N1).

FIG. 13B Hemagglutinin inhibition of pig antisera to FluA virus (H3N2).

FIG. 14 Binding activity of anti-HA mouse/human chimeric mAb hLD9 to Flu Pep06 (HA) and influenza A viruses of Group 1 (H1N1pdm09) and Group 2 (H3N2).

FIG. 15 Binding activity of anti-NA mouse/human chimeric mAb hNB5 to Flu Pep10 (NA) and influenza A virus Group 2 (H3N2).

FIG. 16 Binding activity of anti-Matrix (M1/M2/M2e) mouse/human chimeric mAb hGA4 to Flu Pep5906 (Matrix (M1/M2/M2c)) and influenza A viruses of Group 1 (H1N1pdm09) and Group 2 (H3N2).

FIG. 17 Binding activity of anti-PGN mouse/human chimeric mAb hJG7 to ultrapure PGN from Staphylococcus aureus (SA), ethanol-killed Mycobacterium tuberculosis (MTB), Mycobacterium smegmatis (SMEG), Staphylococcus epidermidis (SE), and SA. All bacteria were ethanol-fixed overnight at room temperature,

FIG. 18 Opsonophagocytic killing activity (OPKA) of anti-PGN mouse/human chimeric mAb hJG7 against SMEG, using a macrophage cell line U-937.

FIG. 19 Binding activity of anti-PGN mouse/human chimeric mAb hMD11 to ultrapure PGN from SA, ethanol-killed MTB, SMEG, SE, and SA. All bacteria were ethanol-fixed overnight at room temperature.

FIG. 20 OPKA of anti-PGN mouse/human chimeric mAb hMD11 against SMEG, using a macrophage cell line U-93

### DESCRIPTION OF THE INVENTION

Vaccinations and vaccines are often the best mechanism for avoiding an infection and preventing the spread of debilitating and dangerous pathogens. With respect to viral infections, parasitic infections and many bacterial infections, vaccinations may be the only effective option as preventative or treatment options are few and those that are available provide only limited effectiveness. Conventional vaccinations require a priori understanding or general identification of the existing antigenic regions of the pathogen. The pathogen itself is propagated and a suitable vaccine developed from heat-killed or otherwise attenuated microorganisms. Alternatively, an antigen or collection of antigens is identified that will generate a protective immune response upon administration. The need for a vaccine is especially urgent with respect to preventing infection by certain bacteria, viruses and parasites. Some bacteria and especially certain viruses mutate constantly or mutate when passing through an intermediate host, often rendering the vaccine developed to the prior or originating bacteria or virus useless against the new strains that emerge. As a consequence, some vaccines may need to be reformulated yearly (or more often) and often administered at fairly high doses. The development and manufacturing costs are high and administering vaccines pose a great many complications and associated risks to patients.

An epitope, also known as an antigenic determinant, is the part of an antigen that is recognized by the immune system such as, for example, by antibodies, B cells, and T cells. This recognition elicits an adaptive immune response. The epitopes of protein antigens are divided into two categories, conformational epitopes and linear epitopes, based on their structure and interaction with the paratope. Conformational and linear epitopes interact with the paratope based on the 3-D conformation adopted by the epitope, which is determined by the surface features of the involved epitope residues and the shape or tertiary structure of other segments of the antigen. A conformational epitope is formed by the 3-D conformation adopted by the interaction of discontiguous amino acid residues. In contrast, a linear epitope is formed by the 3-D conformation adopted by the interaction of contiguous amino acid residues. A linear epitope is not determined solely by the primary structure of the involved amino acids. Residues that flank such amino acid residues, as well as more distant amino acid residues of the antigen affect the ability of the primary structure residues to adopt the epitope's 3-D conformation. Epitopes, as referred to herein, include either or both conformational epitopes and linear epitopes.

Antigens and epitopes as disclosed herein, including specific combinations as described herein, were surprisingly discovered to be effective and in many cases, more effective at relatively low dosages. These antigens contain or are derived from a plurality of antigenic regions (e.g., epitopes which may be continuous or discontinuous epitopes) of a pathogen or of different pathogens. Most all viruses such as Influenza virus and Corona virus, and most all bacterial microbes contain both continuous and discontinuous epitopes. For example, the various strains (e.g., A, B, C, and D; H1-H18 and N1-N11 inclusive) of Influenza virus (e.g., H1N1; H1N9; H3N2; H3N8; H5N2; H7N7; H9N2) contain hundreds of epitopes. Epitopes A198, S199, R201 (H3N2) of influenza virus HA protein are believed to be continuous, whereas other epitopes of HA protein (e.g., G49, K50, L59, D60, 162, D63, P74, H75, V78, F79, R90, K92, F94, P143, D271, P273, 1274, D27; H3N2) are believed to be discontinuous. Epitopes D147, H150, H197, D198, E199, K221, D251 (H3N2) of influenza virus NA (neuraminidase) proteins are believed to be discontinuous, whereas epitopes S367, S372, N400 (H1N9) are believed to be continuous.

Composite antigens or composite epitopes of the invention may contain an antigenic region that represents a combination of all or parts of two or more epitopes (e.g., a composite peptide), or a plurality of immunologically responsive regions (e.g., composite epitopes) derived from one or multiple antigenic sources (e.g., epitopes of viruses, parasites, bacteria, fungi, cells). These immunological regions are amino acid sequences or epitopes that are generally highly conserved sequences found at those antigenic regions of a pathogen or other antigen associated with an infection or a disease or, importantly, associated with stimulation of the immune system to provide protection against the pathogen. Vaccines may be administered via injection (e.g., intramuscular, intradermal, intravenous, intraperitoneal) or taken orally or intranasally. Preferably, immunogenic compositions are administered collectively to animals such as in a water or food supply, or as an aerosol dispensed in a closed or partially closed environment, thereby avoiding the need and expense of providing the vaccine individually.

Composite epitope vaccine antigen sequences are unique peptide antigens that combine conserved peptide sequences from the same, or different microbes into one sequence that provides a peptide that is different from any peptide sequence found in nature. Peptide epitopes may be known or previously unknown epitopes that have been identified in microbes such as bacteria, parasites, fungi, or viruses. One or more epitopes from a single microbe can be sequenced as a single, or repeated epitope and may be combined with one or more epitopes from one or more other pathogens in a continuous peptide sequence. The peptide and/or composite peptide antigens may be to a single microbe or to one or more microbes, or viruses, such as for example, influenza, coronavirus, adenovirus, or respiratory syncytial virus. The peptide and/or composite peptide antigen may also be from a single bacterium, or from one or more gram positive, or gram-negative bacteria, such Pneumococcus spp., Staphylococcus spp. (e.g., S. aureus), Mycobacteria spp. (e.g., M. tuberculosis, M. smegmatis, M. leprae, M. kansasii, M. mantenii, M. fortuitum, or M. xenopi), Bacillus spp. (e.g., B. subtilis), Escherichia spp. (e.g., E. coli), Haemophilus spp. (e.g., H. influenza), Salmonella spp., etc. The epitopes may be combined in any order or configured to provide an immunogenic structure that induces an immune response in a host immunized with the peptide vaccine.

One embodiment of the invention is directed to peptide epitopes of a pathogen, such as viral, parasitic and/or bacterial antigens. Antigens and peptide epitopes disclosed herein may be selected regions of a viral, parasitic, and/or bacterial microbe that is known or believed to generate an effective immune response after administration. The peptide sequence may contain a plurality of immunologically responsive regions or epitopes of one or more pathogens, which are artificially arranged, preferably along a single amino acid sequence or peptide. The plurality may contain multiples of the same epitope, mimotope and/or composite epitope, although generally not in a naturally occurring order, or multiples of a variety of different epitopes from one or more pathogens. Epitopes may be identical to known immunological regions of a pathogen, or entirely new constructs (e.g., mimotopes, composites) that have not previously existed and therefore artificially constructed. Preferably, the antigen of this disclosure induces a protective immunogenic response in the mammal (e.g., human) or an animal and stimulates both mucosal and systemic immune responses similar to those of the natural infection. Preferably that response includes the production of killer T-cell (Tc or CTL) responses, helper T-cell (TH) responses, macrophages (MP), and specific antibody production in an inoculated subject. Also, preferably the response generates antibodies which are positively opsonic in an opsonophagocytic killing assay (OPKA).

Antigens of the invention may also be obtained or derived from the sequences of a pathogen such as, for example, multiple or combined epitopes of the proteins and/or polypeptides of gram-positive and/or gram-negative bacteria, for example, but not limited to Streptococcus, Pseudomonas, Mycobacterium such as M. tuberculosis, Shigella, Campylobacter, Salmonella, Haemophilus influenza, Chlamydophila pneumonia, Corynebacterium diphtheriae, Clostridium tetani, Mycoplasma pneumonia, Staphylococcus aureus, Moraxella catarrhalis, Legionella pneumophila, Bordetella pertussis, Escherichia coli, such as E. coli 0157, and multiple or combined epitomes of conserved regions of any of the foregoing. Exemplary parasites from which sequences may be obtained or derived include but are not limited to Plasmodium such as Plasmodium falciparum and Trypanosoma. Exemplary fungi include, but are not limited to, Aspergillus fumigatus and Aspergillus flavus. Exemplary viruses include, but are not limited to arena viruses, bunyaviruses, coronaviruses, paramyxoviruses, filoviruses, Hepadna viruses, herpes viruses, orthomyxoviruses, orthopneumovirus, parvoviruses, picornaviruses, papillomaviruses, reoviruses, retroviruses, rhabdoviruses, and togaviruses. Preferably, the virus epitopes are obtained or derived from sequences of Influenza viruses.

Antigens as disclosed herein include composite antigens (which contain one or more composite or other epitopes), which are engineered, artificially created antigens made from two or more epitopes, such that the resulting composite antigen has physical and/or chemical properties that differ from or are additive of the individual epitopes. Preferably the composite antigen, when exposed to the immune system of a mammal or other animal, is capable of simultaneously generating an immunological response to each of the constituent epitope of the composite and preferably to a greater degree (e.g., as measurable from a cellular or humoral response to an identified pathogen) than the individual epitopes. In addition, the composite antigen provides the added function of generating a protective immunological response in a mammal or an animal when used as a vaccine and against each of the constituent epitopes. Preferably, the composite has the additional function of providing protection against not only the pathogens from which the constituents were derived, but related pathogens as well. These related pathogenic organisms may be different strains and/or different serotypes of the same species of organism, or different species of the same genus of organism, or different organisms entirely that are only related by a common epitope.

Composite peptides may contain one or more composite epitopes that represent two or more epitopes with epitope sequences only similar to the epitope sequences from which they were derived. Epitopes are regions obtained or derived from a conserved region of a protein or peptide of a pathogen that elicit a robust immunological response when administered to a mammal or an animal. Preferably, that robust response provides the subject with an immunological protection against developing disease from exposure to the pathogen. A preferred example is a composite epitope, which is one artificially created from a combination of two or more highly conserved, although not identical, amino acid sequences of two or more different, but otherwise related pathogens. The pathogens may be of the same type, but of a different strain, serotype, or species or other relation. The composite epitope contains the conserved region that is in common between the related epitopes and also contains the variable regions which differ. Preferably the conserved region contains about 20 or less amino acids on each side of the variable amino acids, preferably about 15 or less, preferably about 10 or less, preferably about 8 or less, preferably about 6 or less, and more preferably about 4 or less. Preferably the amino acids that vary between two similar, but not identical conserved regions are 5 or less, preferably 4 or less, preferably 3 or less, preferably 2 or less, and more preferably only 1.

A “composite epitope,” similar to the composite antigen, is an engineered, artificially created single epitope made from two or more constituent epitopes, such that the resulting composite epitope has physical and/or chemical properties that differ from or are additive of the constituent epitopes. Preferable the composite epitope, when exposed to the immune system of a mammal or an animal, is capable of simultaneously generating an immunological response to each of the constituent epitopes of the composite and preferably to a greater degree than that achieved by either of the constituent epitopes individually. In addition, the composite epitope provides the added function of generating a protective immunological response in a patient when used as a vaccine and against each of the constituent epitopes. Preferably, the composite has the additional function of providing protection against not only the pathogens from which the constituents were derived, but related pathogens as well. These related pathogenic organisms may be strains or serotypes of the same species of organism, or different species of the same genus of organism, or different organisms entirely that are only related by a common epitope.

Composite epitopes of the invention are entirely artificial molecules that do not otherwise exist in nature and to which an immune system has not been otherwise exposed. Preferably, these conserved immunological regions that are combined as a composite epitope represent immunologically responsive regions of proteins and/or polypeptides that are highly conserved between related pathogens. Although a vaccine can be developed from a single composite epitope, in many instances the most effective vaccine may be developed from multiple, different composite epitopes.

Composite antigens of the invention may contain one or more epitopes or composite epitopes, which may include one or more known epitopes to provide an effective vaccine. Although composite antigens may comprise a single composite epitope, a composite antigen would not comprise only a single known epitope. Preferably, the immunological response achieved from a vaccination with a composite antigen, or group of composite antigens, provides protection against infection caused by the original strains from which the sequence of the composite antigen was derived and also provides immunological protection against other strains, serotypes and/or species that share one or more of the general conserved regions represented in the composite antigen. Preferably that response stimulates both mucosal and systemic immune responses in the mammal or the animal, similar to those of the natural infection. Thus, the resulting immune response achieved from a vaccination with a composite antigen is more broadly protective than can be achieved from a conventional single antigen vaccination against multiple strains, serotypes, and species or otherwise related pathogens regardless of antigenic drift that may take place in the evolution of the pathogen. Preferably, vaccines developed from composite antigens of the invention avoid any need for repeated or annual vaccinations, the associated complications and expenses of manufacture, and the elevated risks to the subject. These vaccines are useful to treat individual animals, mammals, and populations or either, thereby preventing infection and mortality and subsequently infections in mammals including pandemics. Such vaccines are also useful to compliment conventional vaccines.

As discussed herein, the antigens disclosed and described herein preferably comprises a single chain of amino acids with a sequence derived from one or more epitopes or a plurality of epitopes, mimotopes, and/or composite epitopes that may be the same or different. Epitope sequences may be repeated consecutively and uninterrupted along a composite sequence or interspersed among other sequences that may be single or a few amino acids as spacers or sequences that encode peptides (collectively spacers), and may be nonimmunogenic or immunogenic and capable of inducing a cellular (T cell) or humoral (B cell) immune response in an animal or a mammal. T-cell stimulating antigens include, for example, tetanus toxin, tetanus toxin heavy chain proteins, diphtheria toxoid (e.g., recombinantly engineered or purified CRM197), tetanus toxoid, Pseudomonas exoprotein A, Pseudomonas aeruginosa toxoid, Bordetella pertussis toxoid, Clostridium perfringens toxoid, Escherichia coli heat-labile toxin B subunit, Neisseria meningitidis outer membrane complex, Hemophilus influenzae protein D, Flagellin Fli C, Horseshoe crab Haemocyanin, and fragments, derivatives, and modifications thereof. Peptide sequences from unrelated microbes may be combined into a single composite antigen. For example, viral sequences of selected immunoresponsive peptides may be interspersed with conserved sequences or epitopes selected from other microbes, such as, for example, bacteria such as M. tuberculosis, S. pneumococcus, P. aeruginosa or S. aureus, viruses such as respiratory viruses, or parasites, such as malaria. Preferred viral proteins, from which preferred epitopes may be selected, include, but are not limited to the influenza virus proteins HA, NA, and M2e, and/or coronavirus proteins spike(S), polymerase (POL), envelope (E), membrane (M), and nucleocapsid (N).

An epitope of the antigen may be of any sequence and size, but is preferable composed of natural amino acids or mimotopes (i.e., a peptide and mimics the structure of an epitope but is composed of a different amino acid sequence than the natural epitope) and is more than 5 but less than 100 amino acids in length, preferably less than 80, preferably less than 70, preferably less than 60, preferably less than 50, preferably less than 40, preferably less than 30, preferably between 5 and 25 amino acids in length, preferably between 8 and 20 amino acids in length, and more preferably between 5 and 15 amino acids in length. Mimotopes may have 1, 2, 3, 4, 5, 6, 7, 8, 9, 10 or more amino acid differences as compared to the natural epitope. Antigens preferably contain any number of epitopes, mimotopes, and/or composite epitopes. The most effective number of epitopes of a antigen against a particular pathogen, pathogen family, or group of pathogens may be determined by one skilled in the art from the disclosures of this application and using routine testing procedures. Antigens may be effective with one epitope, preferably with 2 or more, 3 or more 4 or more, 5 or more or greater. Optionally, antigens may include one or more spacers between epitopes which may be sequences of antigenic regions derived from the same or from one or more different pathogens, or sequences that serve as immunological primers or that otherwise provide a boost to the immune system. That boost may be generated from a sequence of amino acids that are known to stimulate the immune system, either directly or as an adjuvant. Preferred adjuvants comprise analgesic adjuvants, inorganic compounds such as alum, aluminum hydroxide, oil in water emulsion, squalene oil in water nano-emulsion, aluminum phosphate, calcium phosphate hydroxide, mineral oil such as paraffin oil, bacterial products such as killed bacteria Bordetella pertussis, Mycobacterium bovis, toxoids, nonbacterial organics such as squalene, detergents, plant saponins such as Quillaja (Quil A), soybean, Polygala senega, cytokines such as IL-1, IL-2, IL-12, Freund's complete adjuvant, Freund's incomplete adjuvant, food-based oil, Adjuvant 65, which is a product based on peanut oil, and derivatives, modifications and combinations thereof. Preferred adjuvants include, for example, AS01 (Adjuvant System 01) which comprises TLR4 ligand, 3-O-desacyl-4′-monophosphoryl lipid (MPL), and a saponin, QS-21, AS01b which is a component of the adjuvant Shingrix, ALF (Army Liposome Formulation) which comprises liposomes containing saturated phospholipids, cholesterol, and/or monophosphoryl lipid A (MPLA) as an immunostimulant. ALF is safe to use in humans (e.g., has no harmful clinical effects), and increases potency of the vaccine component. ALF modifications and derivatives include, for example, ALF adsorbed to aluminum hydroxide (ALFA), ALF containing QS21 saponin (ALFQ), and ALFQ adsorbed to aluminum hydroxide (ALFQA). A preferred adjuvant formulation comprises a liposome, saponin, lipid A, squalene, unilamellar liposomes having a liposome bilayer that comprises at least one phosphatidylcholine (PC) and/or phosphatidylglycerol (PG), as phospholipids, which may be dimyristoyl phosphatidylcholine (DMPC), dipalmitoyl phosphatidylcholine (DPPC), distearyl phosphatidylcholine (DSPC), dimyristoyl phosphatidylglycerol (DMPG), dipalmitoyl phosphatidylglycerol (DPPG), and/or distearyl phosphatidylglycerol (DSPG), a cholesterol, a monophosphoryl lipid A (MPLA), and a saponin. Preferably the mole ratio of the cholesterol to the phospholipids is greater than about 50:50, and also that the unilamellar liposomes have a median diameter size in micrometer range as detected by light scattering analysis. Additional preferred adjuvants are disclosed in U.S. Pat. No. 10,434,167, which issued Oct. 8, 2019, the entirety of which is incorporated by reference herein.

In one preferred form, antigens useful to generate an immunological response against influenza virus comprise epitopes of HA, M or Matrix (M1, M2, M2e), and/or NA proteins, and/or new epitopes derived from similar conserved regions of different serotypes and strains of influenza virus, and/or from the S or POL protein of coronavirus. Also preferred are antigens epitopes of proteins of Mycobacterium tuberculosis and Clostridium tetani, and/or new epitopes derived from similar conserved regions of different serotypes of these bacteria. Another preferred antigen would include HIV and or malaria epitopes combined with one or more of the above microbial epitopes.

Another form of the antigen comprises a contiguous sequence of one or more epitopes, which may comprise known epitopes, from one or more pathogens in a sequence that does not exist naturally and must be artificially constructed. For example, a contiguous sequence may contain epitopes in closer proximity to each other than would otherwise occur naturally or may contain spacer sequences between epitopes that do not otherwise occur naturally. Preferably, a contiguous sequence of the invention contains one or more epitopes, which is a combination of the sequences of the conserved regions of epitopes that are common to multiple pathogens plus those amino acids that differ between the two conserved regions. For example, where two pathogens contain similar conserved regions that differ by only a single amino acid, the composite sequences would include the conserved region amino acids and each of the amino acids that differ between the two regions as discussed herein.

It is also preferable that a antigen of the invention contain a plurality of repeated epitopes and, optionally, epitopes conjugated with linker regions between or surrounding each epitope, and the plurality of epitopes be the same or different. Preferred linkers include amino acid sequences of antigenic regions of the same or of different pathogens, or amino acids sequences that aid in the generation of an immune response. Preferred examples include, but are not limited to, any of the various antigenic regions of bacteria such as, but not limited to M. tuberculosis, S. aureus and E. coli and viruses such as, but not limited to influenza, coronavirus and HIV and parasites such as P. falciparum. It is also preferred that antigens contain epitopes that generate a systemic and/or a mucosal immune responses similar to that produced from a natural infection.

Another embodiment of the invention is directed to methods for treating or preventing infection of bacteria, virus, parasites, or other microorganisms in a mammal comprising administering to the mammal polyclonal or monoclonal antibodies that are specifically reactive against the peptides disclosed here. Preferably the polyclonal or monoclonal antibodies generate cellular phagocytic activity, destruction of the microorganism, enhances cytokine induced immunity to the microorganism or neutralizes toxic substances of the microorganism, and/or cocktails of two or more monoclonal antibodies (MABs) that enhance immunity to the microorganism. Preferably, the anti-microorganism antibodies are polyclonal antibodies or monoclonal antibodies and react against one or more MTB moieties.

Another embodiment of the invention is directed to monoclonal antibodies that are specifically reactive against epitopes of the microorganism. The part of an antibody that binds to the epitope is referred to as the paratope. Preferably the monoclonal antibody is an IgA, IgD, IgE, IgG or IgM (including subtypes thereof such as, for example, IgG1, IgG2, IgG2a, IgG2b, IgG2c, IgG3 and IgG4), and may be derived from most any mammal such as, for example, human, porcine, caprine, murine, leporidae, muridae, and equine, to include rabbit, guinea pig, mouse, human, fully or partly humanized, chimeric or single chain of any of the above. Preferably monoclonal antibodies of the collection have a normal half-life or have been altered to have an extended half-life on the order of 1.5 times or greater than the half-life of an unaltered antibody. Modifications are preferably through recombinant engineering such as YTE modifications. The DNA encoding the antibodies may be utilized in any appropriate cell line to produce the encoded MABs. Another embodiment comprises hybridoma cultures that produce the monoclonal antibodies. Another embodiment of the invention comprises non-naturally occurring polyclonal antibodies that are specifically reactive against the microorganism. Some important monoclonal antibodies are described in Table 1.

TABLE 1

| Mouse | | | | Hybridoma | Target | mAb |
|---|---|---|---|---|---|---|
| ID | Immunogen | Conjugate | Hybridoma | (mAb) ID | Clone ID | Antigen |
| MS 1435 | TB Pep01 | CRM | LD7 | LD7 I BB2 I | 16kD HSP16.3 | IgG2a |
| | | | | B9 | TB Pep01 | |
| MS 1435 | TB Pep01 | CRM | CA6 | CA6 II GA8 I | 16kD HSP16.3 | IgG2b |
| | | | | A5 | TB Pep01 | |
| MS 190 | Ultrapure | CRM | MD11 | MD11 I C11 | PGN | IgG2b |
| | Peptidoglycan from | | | | | |
| | S. aureus (PGN) | | | | | |
| MS 2209 | A/Wuhan (H3N2) + | CRM | NB5 | NB5 II C2 I K8 | Neuraminidase | IgG2a |
| | Flu Pep11 | | | | (NA) Flu | |
| | | | | | Pep10 | |
| MS 2209 | A/Wuhan (H3N2) + | CRM | LD9 | LD9 III D6 | Hemagglutinin | IgG1 |
| | Flu Pep11 | | | | (HA) Flu | |
| | | | | | Pep06 | |
| MS 2209 | A/Wuhan (H3N2) + | CRM | EA9 | EA9 I F7 | Hemagglutinin | IgG1 |
| | Flu Pep11 | | | | (HA) Flu | |
| | | | | | Pep03 | |
| MS 1443 | Flu Pep5906 | CRM | GA4 | GA4IG11 | Matrix | IgG1 |
| | | | | | (M1/M2/M2e) | |
| | | | | | Flu Pep5906 | |
| MS 2016 | Flu Pep5906 + | CRM | CG6 | CG6 II H8 | Matrix | IgG3 |
| | Flu Pep11 | | | | (M1/M2/M2e) | |
| | | | | | Flu Pep5906 | |
| MS 2016 | Flu Pep5906 + | CRM | KC7 | KC7 I D8 | Matrix | IgG3 |
| | Flu Pep11 | | | | (M1/M2/M2e) | |
| | | | | | Flu Pep5906 | |
| DRAGA5 | Ultrapure | CRM | DRG-5 BD11 | DRG-5 BD11 | PGN | IgM |
| | Peptidoglycan from | | | II E6 II G1 | | |
| | S. aureus (PGN) + | | | | | |
| | TB Pep01 | | | | | |

Hybridoma cell lines that express the monoclonal antibodies disclosed herein were deposited with the American Type Culture Collection (ATCC; Manassas, VA). Hybridomas that produce monoclonal antibodies EA9 (PTA-127659), KC7 (PTA-127660), DRG-5BD11 (PTA-127658), CG6 (PTA-127661), and LD9 (PTA-127662) (as identified in Table 1) were each deposited with ATCC on Oct. 13, 2023. The hybridoma cell lines that express MAB MD11 (PTA-127712), GA4 (PTA-127713), and NB5 (PTA-127714) were deposited with ATCC on Mar. 14, 2024. Monoclonal antibodies produced by these hybridomas may include variable and hypervariable regions, CDR, and Fc regions that may be separately obtained and useful as such. These monoclonal antibodies may be fully or partly humanized, bispecific, and/or conjugated. Another embodiment of the invention is directed to methods for treating or preventing infection by administering a monoclonal or polyclonal antibody that is specifically reactive against the microorganism.

Another embodiment of the invention is directed to method of immunizing mammals or animals with the immunogenic compositions of the invention. Preferably, the vaccines of the invention are less susceptible to variation of antigenicity due to antigenic shift of pathogens which reduces or eliminates the need for annual or repeated vaccination to maintain protection of the mammal or animal populations against potential outbreaks of infection from, for example, new bacterial strain or viral isolates. In addition, the vaccines of the invention generally and advantageously provide increased safety considerations, both in their manufacture and administration (due in part to a substantially decreased need for repeated administration), a relatively long shelf life in part due to minimized need to reformulate due to strain-specific shift and drift, an ability to target immune responses with high specificity for particular microbial epitopes, and an ability to prepare a single vaccine that is effective against multiple pathogens, each of which may be a different. As single immunization to provided protection against one, or more viruses, bacteria, or parasites, such as influenza, coronavirus, HIV, M. tuberculosis, S. aureus, or malaria. The invention encompasses antigenic compositions, methods of making such compositions, and methods for their use in the prevention, treatment, management, and/or prophylaxis of an infection. The compositions disclosed herein, as well as methods employing them, find particular use in the treatment or prevention of viral, bacterial, parasitic and/or fungal pathogenesis and infection using immunogenic compositions and methods superior to conventional treatments presently available in the art. Preferably, vaccinations of immunogenic compositions of antigens disclosed herein provide protection against a pathogenic infection for more than a one-year cycle, which is typical for pathogens such as influenza virus. More preferably, protection is provided for up to 2 years, 5 years, 10 years, 15 years, 20 years, or longer.

These methods can prevent or control infections, such as, for example, an outbreak of viral, parasitic, fungal or bacterial infection, preferably but not limited to an influenza virus, coronavirus, and/or a tuberculosis bacterial infection, in a selected population of animals or mammals. The method includes at least the step of providing an immunologically effective amount of one or more of the disclosed immunogenic or vaccine compositions to a susceptible or an at-risk animal of a population, for a time sufficient to prevent, reduce, lessen, alleviate, control, or delay the outbreak of such an infection in the general population. Preferably, the administration is performed into the water or food supply, or as an aerosol into a closed or semi-closed environment where the animals are maintained, even temporarily maintained.

Another embodiment of the invention is directed to an immunogenic composition comprising nucleic acid sequences that encode protective antigens and/or epitopes against a pathogen. The sequences can be incorporated into a viral vector, suitable for immunizing a mammal. Preferred pathogens include, but are not limited to bacteria, viruses, parasites, fungi and viruses.

In a preferred example, antigens contain a conserved region derived from an influenza virus subtypes (e.g., influenza viruses with varying HA or NA compositions, such as H1N1, H5N1, H3N2, and H2N2). Epitopes of conserved regions on NA or HA may also confer cross-subtype immunity. As an example, conserved epitopes on NA (N1) may confer enhanced immunity to H5N1 and H1N1. With respect to similar or homologous chemical compounds among influenza A subtypes and/or strains within a subtype, preferably these are at least about 80 percent, more preferably at least about 90 percent, more preferably at least about 95 percent identical, more preferably at least about 96 percent identical, more preferably at least about 97 percent identical, more preferably at least about 98 percent identical, more preferably at least about 99 percent identical, and even more preferably 100 percent identical (invariant). Preferably, at least one peptide sequence within the antigen is also conserved on homologous proteins (e.g., protein subunits) of at least two viral particles, preferably influenza particles. Proteins of influenza virus include, for example, expressed proteins in the virus structure, such as HA, NA, protein polymerases (PB1, PB2, PA), matrix proteins (M1, M2), and nucleoprotein (“NP”). Preferably, the conserved peptide sequences are conserved on at least two or more of the M1, M2, HA, NA, or one or more polymerase proteins.

In a preferred example, a selected sequence in the M1 and M2 proteins of the H5N1 influenza virus corresponds to the M1 and M2 proteins found in other H5N1 particles, and to the same sequence in the M1 and M2 proteins of the H3N2 influenza virus. In addition, while HA and NA proteins have highly variable regions, conserved sequences from HA and NA are found across many influenza strains and many subtypes (e.g., HA and NA sequences are conserved across H5N1 and H1N1). In a preferred embodiment of the invention, the sequences are derived from a conserved sequence present within variants or strains (viral isolates expressing substantially the same HA and NA proteins, but wherein the HA and NA protein amino acid sequences show some minor drift), of a single influenza virus subtype and more preferably across at least two influenza virus subtypes, e.g., subtypes of influenza A virus.

A peptide or polypeptide that includes at least one conserved epitope sequence, which may also comprise one or more repeats of the same or a different epitope sequence, each of which is conserved across a plurality of homologous proteins that is conserved in a population of bacterial, parasitic or viral strains or serotypes, and a pharmaceutically acceptable carrier. In exemplary antigens, at least one epitope sequence (continuous or discontinuous) may be repeated at least once or multiple times. Compositions may include a pharmaceutically acceptable carrier.

Peptide sequences preferably include sequences derived from genome (i.e., RNA) segment 7 of the influenza virus, while in a more preferred embodiment, the sequences include at least portions of the M1 and M2 proteins. In other preferred embodiments, the sequences include sequences expressed from genome segments encoding the HA or NA proteins. Such sequences are less affected by subtype drift and more broadly protective against infections.

Antigens may include one or more T-cell stimulating epitopes, such as diphtheria toxoid, tetanus toxoid, a polysaccharide, a lipoprotein, or a derivative or any combination thereof (including fragments or variants thereof). Typically, at least one repeated sequence of the antigen is contained within the same molecule as the T-cell stimulating epitopes. In the case of protein-based T-cell stimulating epitopes, the at least one repeated sequence of the antigen may be contained within the same polypeptide as the T-cell stimulating epitopes, may be conjugated thereto, or may be associated in other ways. Preferably, one or more T-cell stimulating epitopes are positioned at either the N-Terminus or the C-Terminus (or both) of the antigen.

In additional embodiments, the antigens, with or without associated T-cell stimulating epitopes may include one or more polysaccharides or portions thereof, or one or more or multiple portions of a protein or substantially all of the immunogenic portions of a protein, wherein substantially all means sufficient to treat or prevent an infection. A preferred composition includes an immunogenic portion of a composition comprising an immunogenic portion of a peptidoglycan and an immunogenic portion of a heat shock protein. Preferably, the immunogenic portion of the peptidoglycan is obtained from a gram-positive microorganism and the gram-positive microorganism is of a spp. of Mycobacteria, a spp. of Staphylococcus, a spp. of Bacillus, or a spp. of Streptococcus. Preferably, the immunogenic portion of the peptidoglycan comprises multiple immunogenic portions of a peptidoglycan molecule such as substantially all of the peptidoglycan molecule. Preferably, the immunogenic portion of the heat shock protein is of a spp. of Mycobacteria such as, for example, a spp. of Mycobacteria such as M. tuberculosis, M. smegmatis, M. leprae, M. kansasii, M. mantenii, M. fortuitum, or M. xenopi. Preferably, and further the immunogenic portion is an alpha helix portion of the heat shock protein. Preferably, the immunogenic portion of peptidoglycan and the immunogenic portion of the heat shock protein are a contiguous amino acid sequence. Preferably, the composition includes an adjuvant which is preferably a nano-emulsion. Preferably the composition treats or prevents a gram-positive infection (e.g., Mycobacterial infection) in a mammal and may be a vaccine administered as described herein and induces opsonophagocytic killing activity against a microorganism.

In preferred embodiments, at least one sequence of a antigen is conjugated to one or more polysaccharides. In other embodiments, one or more polysaccharides are conjugated to other portions of the antigen. Certain embodiments of the present invention are selected from polysaccharide vaccines, protein-polysaccharide conjugate vaccines, protein vaccines, or combinations thereof.

Antigens of the invention may be synthesizing by in vitro chemical synthesis, solid-phase protein synthesis, and in vitro (cell-free) protein translation, or recombinantly engineered and expressed in bacterial cells, fungi, insect cells, mammalian cells, virus particles, yeast, and the like.

A antigen may include one of the following elements: at least one repeated epitope; at least one T-cell epitope; at least one polysaccharide (sugar); at least one structural component; or a combination thereof. The one structural component may include one or more of: at least one linker segment; at least one sugar-binding moiety; at least one nucleotide-binding moiety; at least one protein-binding moiety; at least one enzymatic moiety; or a combination thereof. The invention encompasses methods of preparing an immunogenic composition, preferably a pharmaceutical composition, more preferably a vaccine, wherein a target antigen of the present invention is associated with a pharmaceutically acceptable diluent, excipient, or carrier, and May be used with most any adjuvant, such as, for example, ALFQ, ALFQA, ALFA, AS01, AS01b, and/or combinations, derivatives, and modifications thereof.

Within the context of the present invention, that a relatively small number of conservative or neutral substitutions (e.g., 1 or 2) may be made within the sequence of the antigen or epitope sequences disclosed herein, without substantially altering the immunological response to the peptide. In some cases, the substitution of one or more amino acids in a particular peptide may in fact serve to enhance or otherwise improve the ability of the peptide to elicit a systemic response in an animal or a mammal that has been provided with a composition that comprises the modified peptide, or a polynucleotide that encodes the peptide. Suitable substitutions may generally be identified using computer programs and the effect of such substitutions may be confirmed based on the reactivity of the modified peptide with antisera and/or T-cells. Accordingly, within certain preferred embodiments, a peptide for use in the disclosed diagnostic and therapeutic methods may comprise a primary amino acid sequence in which one or more amino acid residues are substituted by one or more replacement amino acids, such that the ability of the modified peptide to react with antigen-specific antisera and/or T-cell lines or clones is not significantly less than that for the unmodified peptide.

As described above, preferred peptide variants are those that contain one or more conservative substitutions. A “conservative substitution” is one in which an amino acid is substituted for another amino acid that has similar properties, such that one skilled in the art of peptide chemistry would expect the secondary structure and hydropathic nature of the peptide to be substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Examples of amino acid substitutions that represent a conservative change include: (1) replacement of one or more Ala, Pro, Gly, Glu, Asp, Gln, Asn, Ser, or Thr; residues with one or more residues from the same group; (2) replacement of one or more Cys, Ser, Tyr, or Thr residues with one or more residues from the same group; (3) replacement of one or more Val, Ile, Leu, Met, Ala, or Phe residues with one or more residues from the same group; (4) replacement of one or more Lys, Arg, or His residues with one or more residues from the same group; and (5) replacement of one or more Phe, Tyr, Trp, or His residues with one or more residues from the same group. A variant may also, or alternatively, contain non-conservative changes, for example, by substituting one of the amino acid residues from group (1) with an amino acid residue from group (2), group (3), group (4), or group (5). Variants may also (or alternatively) be modified by, for example, the deletion or addition of amino acids that have minimal influence on the immunogenicity, secondary structure and hydropathic nature of the peptide.

Epitopes may be arranged in any order relative to one another in the sequence which may be with or without spacers. The number of spacer amino acids between two or more of the epitopic sequences can be of any practical range, including, for example, from 1 or 2 amino acids to 3, 4, 5, 6, 7, 8, 9, or even 10 or more amino acids between adjacent epitopes.

Another embodiment of the invention is directed to polynucleotides including DNA, RNA (e.g., cRNA, mRNA), and PNA (peptide nucleic acid) constructs that encode the sequences of the invention. These polynucleotides may be single-stranded (coding or antisense) or double-stranded, and may be DNA (genomic, cDNA or synthetic) or RNA molecules. Additional coding or non-coding sequences may, but need not, be present within a polynucleotide of the present invention, and a polynucleotide may, but need not, be linked to other molecules and/or support materials. As is appreciated by those of ordinary skill in the art that, as a result of the degeneracy of the genetic code, there are many nucleotide sequences that encode a given primary amino acid sequence. Some of these polynucleotides bear minimal homology to the nucleotide sequence of any native gene. Nonetheless, polynucleotides that vary due to differences in codon usage are specifically contemplated by the present invention. Polynucleotides that encode an immunogenic peptide may generally be used for production of the peptide, in vitro or in vivo. Any polynucleotide may be further modified to increase stability in vivo. Possible modifications include, but are not limited to, the addition of flanking sequences at the 5′ and/or 3′-ends; the use of phosphorothioate or 2′-o-methyl rather than phosphodiesterase linkages in the backbone; and/or the inclusion of nontraditional bases such as inosine, queosine and wybutosine, as well as acetyl-methyl-, thio- and other modified forms of adenine, cytidine, guanine, thymine and uridine.

A nucleic acid vaccine of the invention contains the genetic sequence of a antigen as CRNA or mRNA, or DNA, plus other necessary sequences that provide for the expression of the antigen in cells. By injecting the mammal with the genetically engineered nucleic acid, the antigen is produced in or preferably on cells, which the mammal's immune system recognizes and thereby generates a humoral or cellular response to the antigen, and therefore the pathogen. Nucleic acid vaccines have a number of advantages over conventional vaccines, including the ability to induce a more general and complete immune response in the mammal. Accordingly, nucleic acid vaccines can be used to protect an animal or a mammal against disease caused from many different pathogenic organisms of viral, bacterial, and parasitic origin as well as certain tumors.

Nucleic acid vaccines typically comprise a viral or bacterial nucleic acid (e.g., cRNA, mRNA, DNA) that encodes an antigen contained in vectors or plasmids that have been genetically modified to transcribe and translate the antigenic sequences into specific protein sequences derived from a pathogen. By way of example, the nucleic acid vaccine is administered, and the cellular machinery transcribed and/or translates the nucleic acid into the antigens which produce an immune response. The antigens, being non-natural and unrecognized by the mammalian immune system, are processed by cells and the processed proteins, preferably the epitopes, displayed on cell surfaces. Upon recognition of these antigens as foreign, the immune system generates an appropriate immune response that protects from the infection. In addition, nucleic acid vaccines of the invention are preferably codon optimized for expression in the animal (or mammal) of interest. In a preferred embodiment, codon optimization involves selecting a desired codon usage bias (the frequency of occurrence of synonymous codons in coding DNA) for the particular cell type so that the desired peptide sequence is expressed.

Compositions of the invention may contain antigens and epitopes, mimotopes, composite sequences, and/or RNA and/or DNA vaccines. Composition may include adjuvants such as, for example, oil in water emulsion, ALFQ, ALFQA, ALFA, AS01, AS01b, and/or combinations, derivatives, and modifications thereof. The formulation of pharmaceutically-acceptable excipients and carrier solutions is well known to those of ordinary skill in the art, as is the development of suitable dosing and treatment regimens for using the particular compositions described herein in a variety of treatment regimens.

The amount of immunogenic composition(s) and the time needed for the administration of such immunogenic composition(s) will be within the purview of the ordinary-skilled artisan having benefit of the present teachings. The administration of a therapeutically-effective, pharmaceutically-effective, and/or prophylactically-effective amount of the disclosed immunogenic compositions may be achieved by a single administration. Alternatively, in some circumstances, it may be desirable to provide multiple, or successive administrations of the immunogenic compositions, either over a relatively short, or even a relatively prolonged period of time, as may be determined by the skilled person overseeing the administration of such compositions.

The immunogenic compositions and vaccines of the present invention preferably contain an adjuvant such as oil in water emulsion or ALFQ and may be given by IM, SQ, Intradermal or intranasal administration or in a manner compatible with the dosage formulation, and in such an amount as will be prophylactically or therapeutically effective and preferably immunogenic. The quantity to be administered depends on the subject to be treated, including, e.g., the capacity of the immune system to mount an immune response, and the degree of protection desired. Suitable dosage ranges may be on the order of several hundred micrograms (μg) of active ingredient per animal or mammal with a preferred range from about 0.1 μg to 2000 μg (even though higher amounts, such as, e.g., in the range of about 1 to about 10 mg are also contemplated), such as in the range from about 0.5 μg to 1000 μg, preferably in the range from about 1 μg to about 500 μg and especially in the range from about 10 μg to about 100 μg. Suitable regimens for initial administration and booster shots are also variable but are typified by an initial administration followed by optional but preferred subsequent inoculations or other periodic administrations.

An effective dose comprises amounts the range of about 0.1 μg to about 1 mg total protein or target antigen per animal or mammal. In one exemplary embodiment, the vaccine dosage range is about 0.1 μg to about 10 mg per animal or mammal. It was surprisingly discovered that the more effective dosages of the various peptide antigens are lower that observed at dosages of 50 μg, at 100 μg, and at higher amounts per animal.

An immunologically effective dose is one that stimulates the immune system of the animal or mammal to establish an immune response to the immunogenic composition or vaccine. Preferably, a level of immunological memory sufficient to provide long-term protection against disease caused by microbial infection is obtained. The immunogenic compositions or vaccines of the invention may be preferably formulated with an adjuvant. By “long-term” it is preferably meant over a period of time of at least about 6 months, over at least about 1 year, over at least about 2 to 5 or even at least about 2 to about 10 years or longer. Preferably protection is provided with one administration (or one initial series of administrations) and multiple administrations over time are not required.

Another embodiment of the invention comprises containers containing one or more effective doses of an immunological composition disclosed herein. Container may be vials of glass or plastic and are preferable free of preservatives and contaminant that may leach from containers into the composition over time. Containers may contain a single dose of the immunogenic composition or multiple doses to be partitioned into individual doses. Depending on the route of administration, the container may be a syringe (e.g., for parenteral such as IV, ID, SubQ or IM injection), a spray bottle (e.g., for nasal administration), a pill or tablet for parenteral administration, or a liquid or powder to be added to livestock feed for oral administration to large number of animal subjects or distribution to wildlife. Preferably the immunogenic composition is a liquid or a lyophilized powder. The immunogenic composition may contain an adjuvant, such as disclosed here or as otherwise known to those skilled in the art, and/or a pharmaceutically acceptable carrier such as, for example, an aqueous substance (e.g., water, buffers) and/or a non-aqueous substance (e.g., oils, lipids). Preferably the immunogenic compositions of disclosed herein are administered as a single dose in the form of a vaccination. Booster doses may be advantageous for certain individuals.

It was determined that lower doses, preferably dosages from 0.01 μg to 0.1 μg, from 0.1 μg to 1 μg, from 1 μg to 2 μg, from 2 μg to 3 μg, from 3 μg to 4 μg, from 4 μg to 5 μg, from 5 μg to 6 μg, from 6 μg to 7 μg, from 7 μg to 8 μg, from 8 μg to 9 μg, or from 9 μg to 10 μg per human or animal subject were more effective than higher doses. However, one may prefer to adjust dosage based on the amount of peptide delivered. In either case, these ranges are merely guidelines from which one of ordinary skill in the art may deviate according to conventional dosing techniques. Precise dosages per subject individual can be determined by assessing the immunogenicity of the conjugate produced in the appropriate host so that an immunologically effective dose is delivered, which was surprisingly demonstrated to be 10 μg or less herein and more effective than higher doses per animal. With human subjects and small numbers of animals subjects, preferably immunogenic compositions are stored and administered in single dose forms. For treatment of large numbers of animal subjects, such as distributions to herds of farm animals or wildlife, administration may be from containers of multiple dose forms.

The following examples illustrate embodiments of the invention but are not to be viewed as limiting the scope of the invention.

### EXAMPLES

### Example 1—Neutralizing Monoclonal Antibodies Raised Against Highly Conserved Influenza Hemagglutinin, Neuraminidase, and Matrix Epitopes May Offer Novel Cocktail Therapeutic Strategies

Clinical management of seasonal influenza outbreaks and pandemics continue to present significant challenges in public health due to circulation of rapidly evolving influenza strains. For individuals with increased risk of influenza exposure, immunosuppressed conditions, or severe disease, the emergence of variants resistant to antiviral therapeutics poses a major threat to their lives. Previous studies investigated therapeutic approaches of broadly reactive monoclonal antibodies (mAbs) directed against composite peptides derived from hemagglutinin (HA), neuraminidase (NA), and matrix (M1/M2/M2e) viral surface proteins. Here, binding was evaluated and functional capabilities of four different isotype specific anti-influenza mAbs analyzed to determine their candidacy for a cocktail therapeutic strategy.

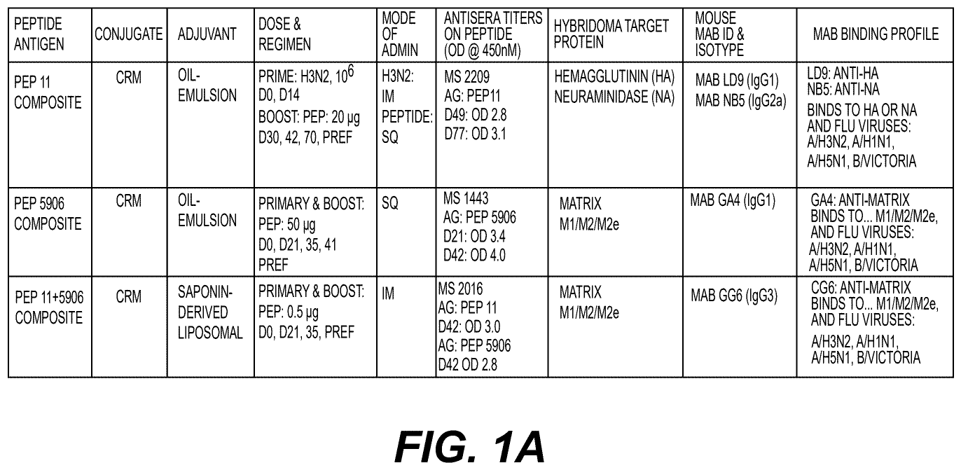

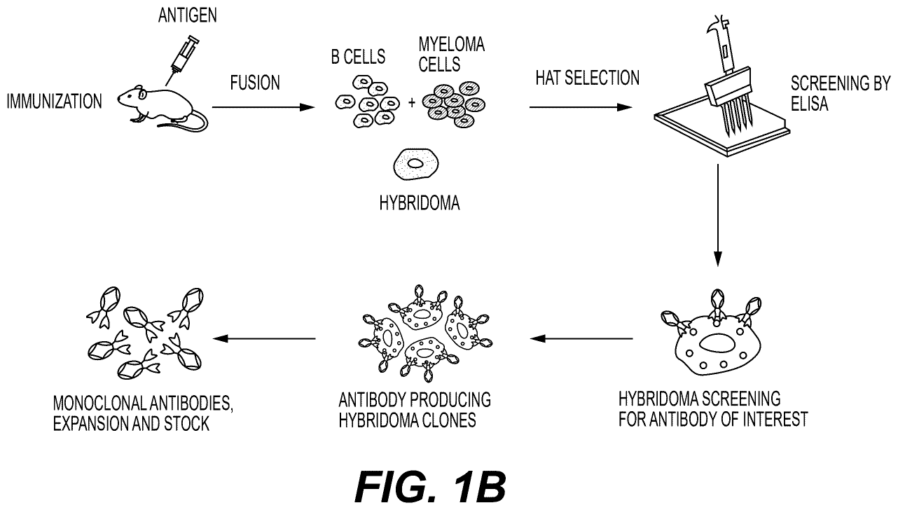

Briefly, mice were immunized as indicated in FIG. 1A, B cells were removed and fused with myeloma cells and subjected to HAT selection to identify fused cells, screened by ELISA, screened again for antibodies of interest, antibody producing cells identified cloned, and clones of interest were identified and expanded (see FIG. 1B).

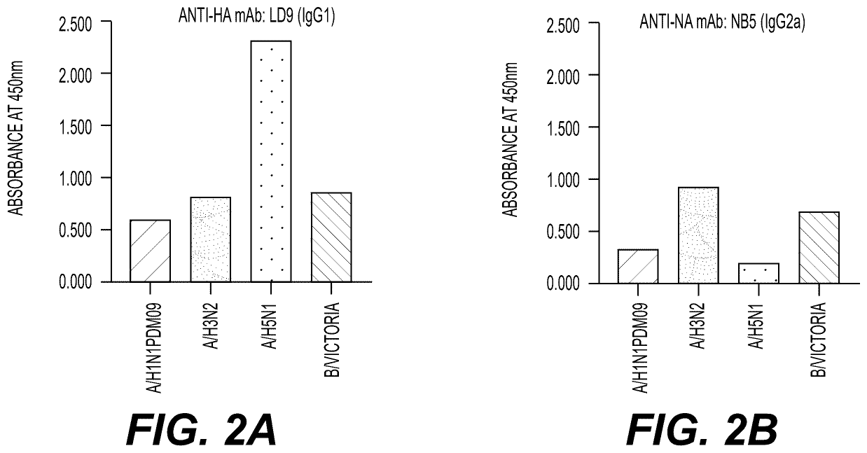

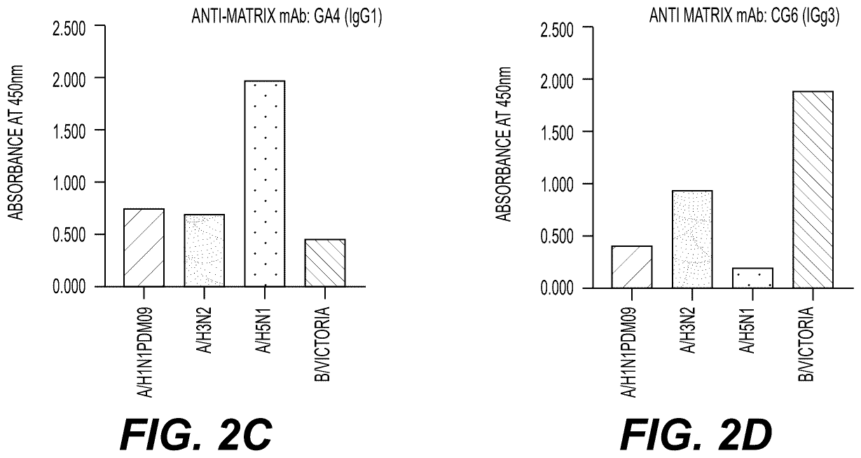

Four mAbs (LD9, NBS, GA4, and CG6) were produced from mice immunized with small composite influenza peptides comprising highly conserved HA, NA, and Matrix (M1/M2/M2e) epitopes. Binding activities of mAbs at 25 μg/mL to live influenza A and B viruses were examined by ELISA (see FIGS. 2A, 2B, 2C, and 2D). Functional capabilities were assessed in vitro using the microneutralization assay. IgG1 anti-HA mAb LD9 binding (FIG. 2A), IgG1 anti-matrix mAb GA4 binding (FIG. 2B), IgG3 anti-matrix mAb CG6 (FIG. 2C), and binding activity to Influenza A and Influenza B viruses (FIG. 2D).

TABLE 2
Virus Annotation

| | |
|---|---|
| Live influenza A/Michigan/45/2015 (H1N1)pdm09 | A/H1N1pdm09 |
| Live influenza A/California/04/2009 (H1N1)pdm09 | A/California/ |
| | H1N1pdm09 |
| Live influenza A/Hong Kong/4801/14 (H3N2) | A/H3N2pdm09 |
| BPL-inactivated influenza A/India/NIV/2006 | A/H5N1 |
| (H5N1) -PR8-IBCDC-RG7 | |
| Live influenza B/New Hampshire/01/2021 (Victoria | B/Victoria |
| lineage) | |

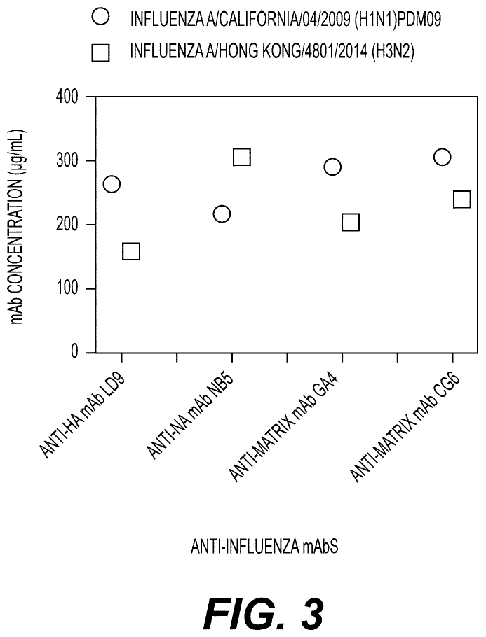

Three peptide antigens (Pep 11; Pep 5906; and Pep11/5906), each conjugated to CRM, were tested. Pep 11 plus an oil emulsion as adjuvant was administered to mice as follows: Primary: H3N2, 10⁶ with DO and D14 boost at 20 μg and D30, D42, D70 and preF. Mode of administration was H3N1 IM and peptide SQ. Antisera titers on peptide (OD at 450 nM) were MS 2209, Ag Pep11 Day 49 at 2.8 and Day 77 at 3.1. Hybridoma target proteins were HA with mAb LD9 (IgG1), and NA with NBS (IgG2a), which bind to Flu viruses: A/H3N2; A/H1N1; A/H5N1, and B/Victoria. Pep 5906 plus an oil emulsion as adjuvant was administered to mice as follows: Primary and Boost: 50 μg with DO, D21, D35 and D41 preF. Mode of administration was SQ. Antisera titers on peptide (OD at 450 nM) were MS 1443, Ag Pep5906, Day 21 at 3.4 and Day 42 at 4.0. Hybridoma target proteins were matrix proteins M1, M2, and M2e with mAb GAA (IgG1), which bind to M1/M2/M2e and Flu viruses: A/H3N2; A/H1N1; A/H5N1, and B/Victoria. Pep 11/5906 plus saponin derived liposomal adjuvant was administered to mice as follows: Primary and Boost: 0.5 μg with DO, D21, and D35 preF. Mode of administration was IM. Antisera titers on peptide (OD at 450 nM) were MS 2106, Ag Pep11, Day 42 at 3.0 and Ag 5906 Day 42 at 2.8. Hybridoma target proteins were matrix proteins M1, M2, and M2e with mAb CG6 (IgG3), which bind to M1/M2/M2e and Flu viruses: A/H3N2; A/H1N1; A/H5N1, and B/Victoria (see FIG. 3).

Anti-influenza mAbs: LD9 (IgG1, anti-HA), NB5 (IgG2a, anti-NA), GA4 (IgG1, anti-matrix), and CG6, (IgG3, anti-Matrix) bound equally well to H3N2 but differentially to other contemporary and pandemic strains. Anti-HA mAb LD9 and anti-Matrix mAb GA4 (both IgG1) preferentially bound to pandemic H5N1 influenza strain; while anti-Matrix mAb CG6 was more reactive with influenza B. Anti-NA mAb NB5 and anti-Matrix mAb CG6 both had higher affinities to H3N2 and influenza B but reacted less well to H1N1 and H5N1. Neutralizing activity of all four mAbs was demonstrated against H1N1 and H3N2.

Mouse anti-influenza mAbs LD9, NB5, GA4 and CG6 bound to influenza A and B viruses, albeit differentially and demonstrated neutralising activity across Group 1 and Group 2 contemporary influenza strains. The preferential mAb binding to influenza virus epitopes might differentiate specificity. Cocktails comprising a mixture of such mAbs offer important strategies for prevention and treatment of influenza, particularly for immunocompromised individuals.

These data indicate that the cocktail of anti-HA mAbs or anti-NA mAbs with anti-matrix mAbs enhances mAb neutralization of influenza viruses (CPE in at least 3 out of 4 wells).

### Example 2—HSP16.3 and Peptidoglycan Cell Wall Components Recognise and Effectively Bind to Clinical Strains of Mycobacterium tuberculosis

Mycobacterium tuberculosis (MTB) strains are increasingly becoming resistant to antibiotics and are among high priority pathogens that contribute to antimicrobial resistance (AMR), thus posing a major global threat to public health. The role of monoclonal antibodies (mAbs) in mediating MTB in vitro opsonization, phagocytosis and killing, and MTB elimination in vivo are known to be highlighted. mAbs raised against targets common to gram-positive bacteria are believed useful for combating AMR and providing novel treatment options for tuberculosis (TB). One such target, heat shock protein (HSP16.3), plays an important role in the persistence of MTB during latent infection while the other target, peptidoglycan (PGN), is an important cell wall component of gram-positive bacteria. In this study, the binding capabilities of IgG2a (anti-HSP16.3) and IgG2b (anti-PGN) mAbs to clinical MTB isolates were analyzed.

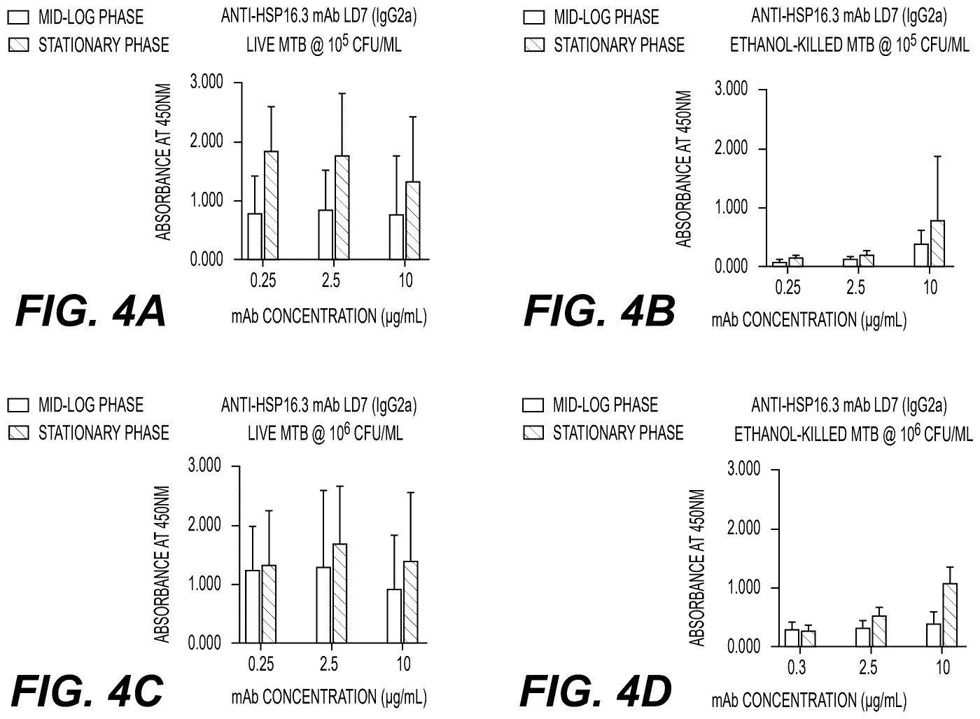

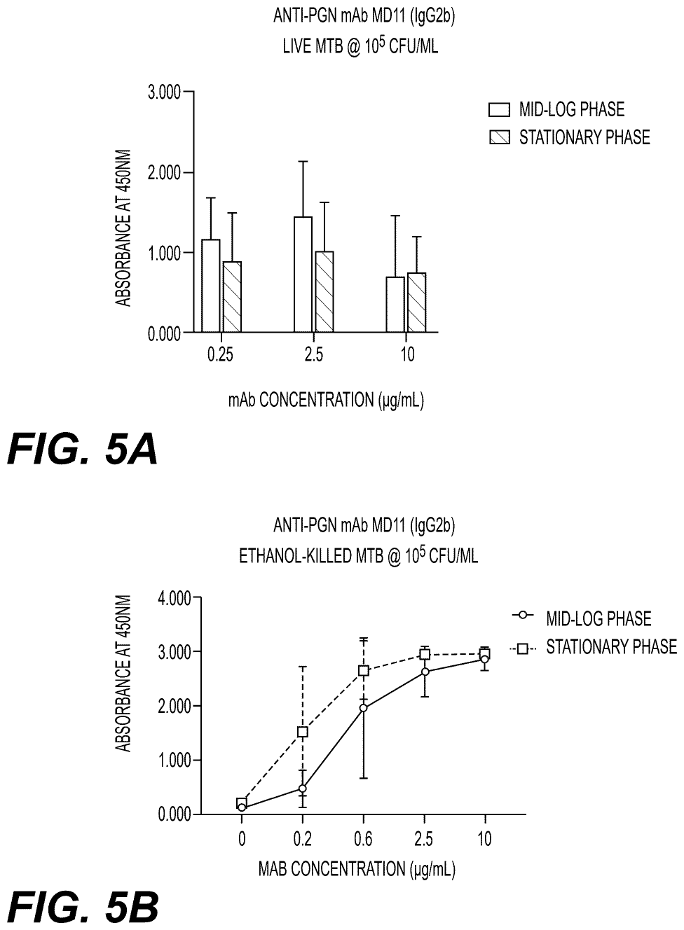

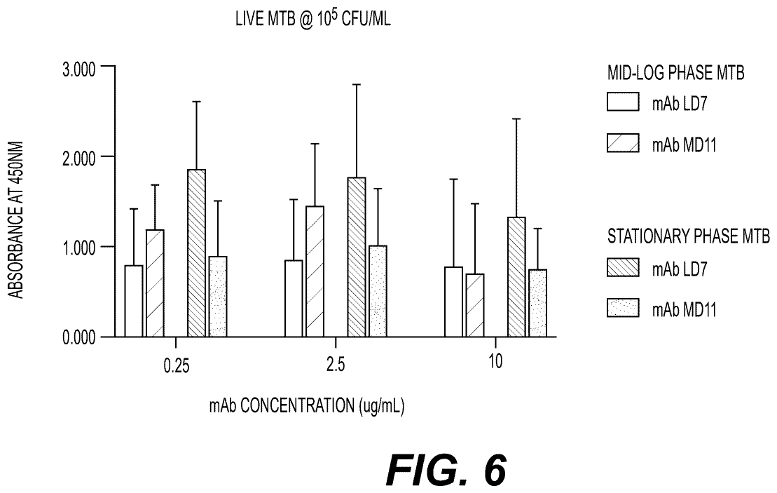

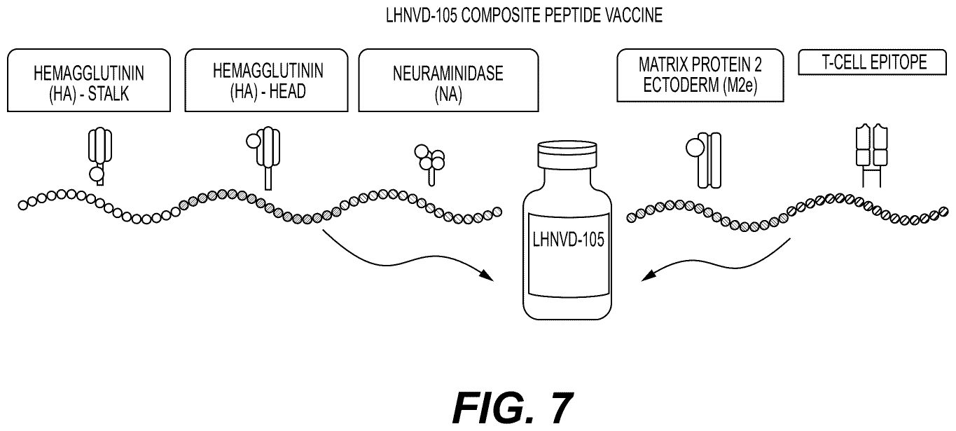

IgG2a and IgG2b mAbs were evaluated for binding activity to clinical MTB; a range of mAb concentrations that constituted low to high levels (0.25, 1, 2.5, 5, 10, 25 μg/mL) was used (see FIGS. 4A, 4B, 4C, and 4D). Live and ethanol-killed MTB were investigated at midlogarithmic/stationary phase (between 104 and 106 CFU/mL) and screened by enzyme-linked immunosorbent assays (ELISAs). Anti-HSP16.3 mAb LD7 (IgG2a) demonstrated good binding activity to live MTB at mid-logarithmic and stationary phases of MTB growth, with bacteria at 106 and 105 CFU/mL (FIGS. 4A and 4C). Anti-HSP16.3 mAb LD7 bound to ethanol-killed MTB at mAb concentrations >10 μg/mL (FIGS. 4B and 4D). Anti-PGN mAb MD11 (IgG2b) bound comparably well to 105 live MTB at mid-logarithmic and stationary phases of MTB growth (FIG. 5A). Anti-PGN mAb MD11 bound strongly to ethanol-killed MTB and showed a dose response with increasing concentration of mAb (FIG. 5B). Anti-HSP16.3 mAb LD7 demonstrated enhanced binding to stationary phase live MTB, while anti-PGN mAb MD11 showed enhanced binding to mid-log phase and stationary MTB growth phases (FIG. 6).

Good binding activity of novel IgG2a and IgG2b mAbs to native epitopes on live and killed clinical MTB was demonstrated at concentrations as low as 0.25 μg/mL. Binding and opsonophagocytic activity of these mAbs were shown against clinical MTB strains (including resistant MTB strains). These mAbs offer a therapeutic approach against TB and provide important strategies to augment treatment of antibiotic resistant bacteria.

### Example 3—Adjuvanted Unconjugated Highly Conserved Multi-Epitope Influenza Peptide Vaccine Induced Broadly Reactive Antibodies to Multiple Influenza Viruses in Pigs

Influenza is a prevalent zoonotic respiratory virus and in swine remains a public health concern because of its capability to generate new reassortants of human, avian, and swine influenza subtypes with pandemic potential. Influenza in pigs can cause acute respiratory disease that results in high morbidity and a reduction in growth performance leading to significant production losses. Due to the rapidly evolving nature of influenza, the swine industry faces many challenges when considering an effective vaccine strategy. Peptides provide a cost-effective and easily scalable strategy towards a universal influenza vaccine. An influenza multi-epitope peptide vaccine generated antibody responses to influenza virus and showed HA/HI activity in pigs would be advantageous. Herein, unconjugated multi-epitope peptides formulated with ADDAVAX™ adjuvant induced serum antibodies to multiple influenza viruses when administered to pigs at low doses.

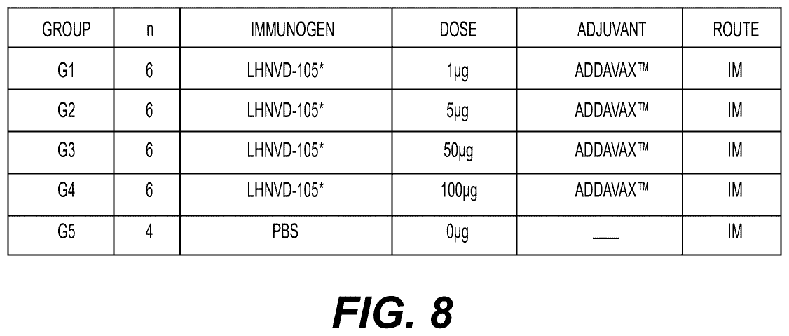

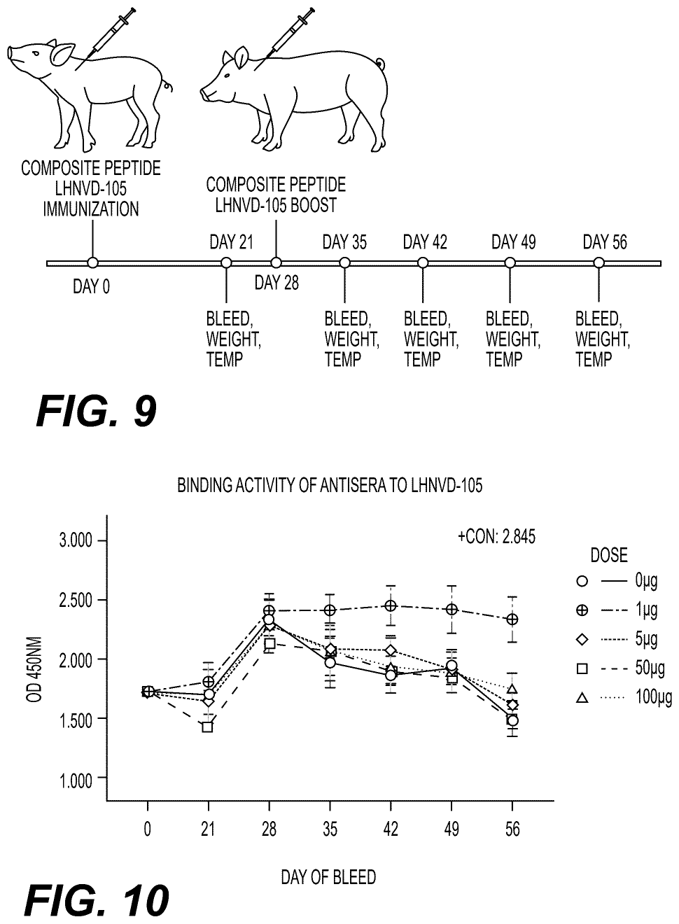

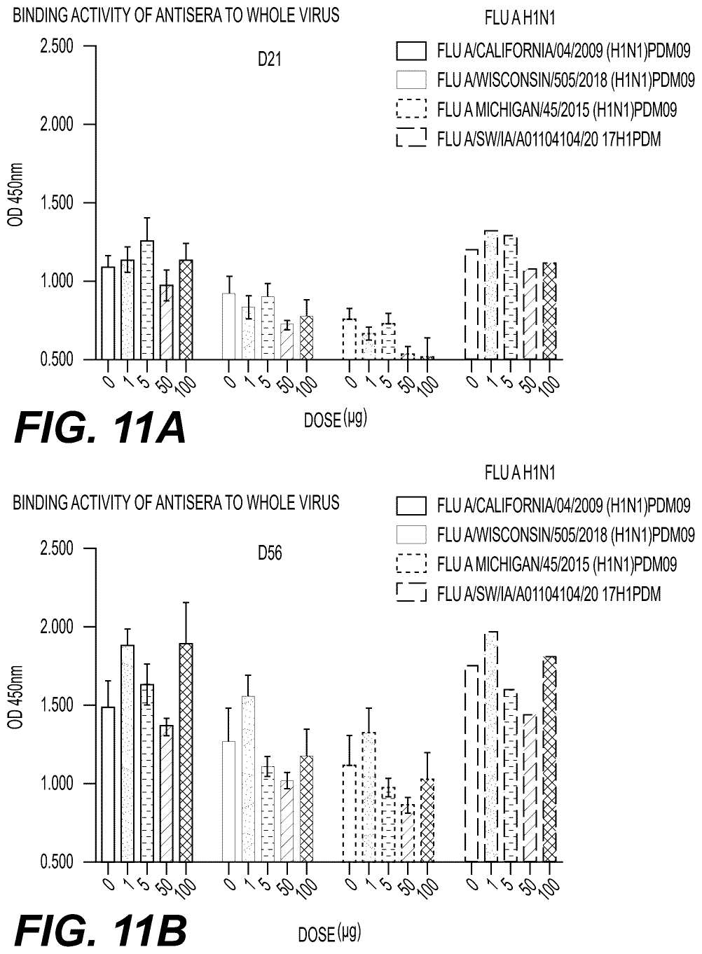

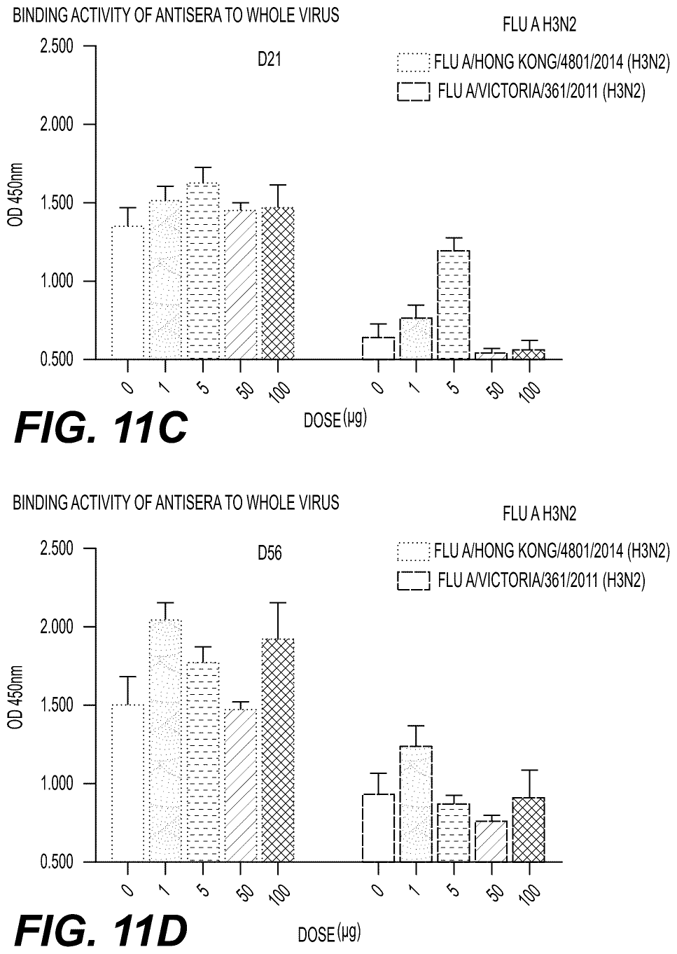

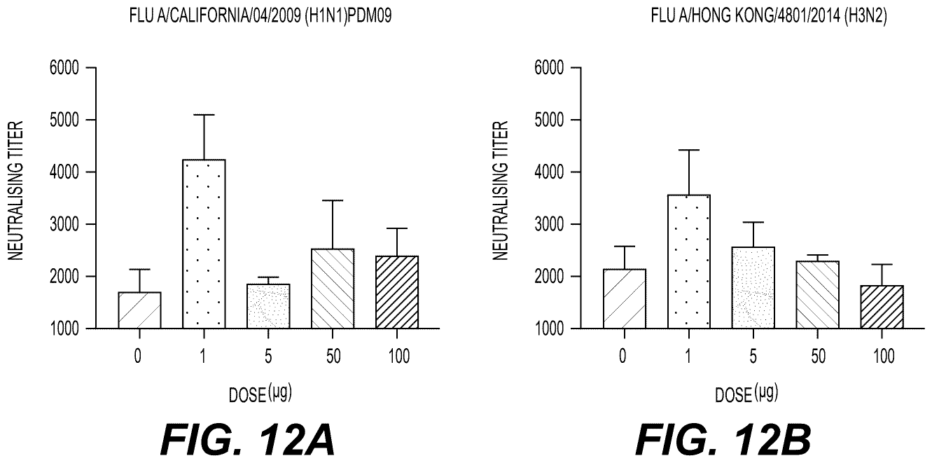

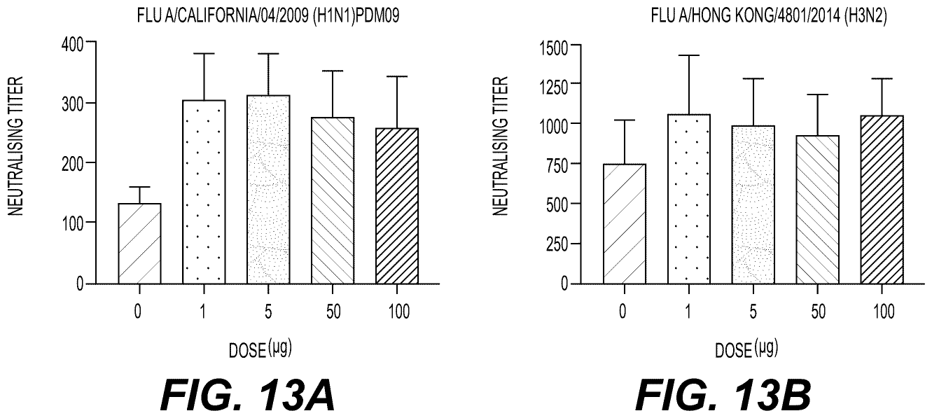

LHNVD-105 is an unconjugated composite peptide vaccine targeting multiple conserved influenza epitopes from hemagglutinin (HA), neuraminidase (NA), and matrix (M1/M2/M2e) protein and includes a universal T-cell epitope. The adjuvate used for all experiments was ADDAVAX™ and the route of administration was IM. Each group consisted of 6 pigs with a control group administered PBS (see FIG. 8). Pigs were immunized intramuscularly on d0 and d28 with LHNVD-105, unconjugated composite influenza peptide vaccine comprising HA+NA and Matrix (M1/M2/M2e)+T-cell epitope, at 1 μg, 5 μg, 50 μg, and 100 μg doses formulated with the squalene-based ADDAVAX™ adjuvant (see FIG. 9). No adverse reactions were observed post immunization in any treatment groups. Serum antibody responses to LHNVD-105 and multiple strains of whole influenza virus were analyzed using ELISA. Antisera functional activity was assessed using microneutralization and hemagglutinin inhibition assay (HAI). Binding activity of antisera from pigs immunized with LHNVD-105 at a 1 μg, 5 μg, 50 μg or 100 μg dose or PBS (0 μg) on do and d28 (arrows) to LHNVD-105 (FIG. 10). Serum antibody responses to various whole H1N1 influenza A (FIGS. 11A and 11B), H3N2 influenza A virus, and influenza B virus (FIGS. 11C and 11D) were analyzed using IgG detection antibody. Data are represented as means±SEM. Day 56 antisera from pigs immunized with LHNVD-105 at a 1 μg, 5 μg, 50 μg or 100 μg dose or PBS (0 μg) on do and d28 (arrows) to LHNVD-105 demonstrated neutralization (FIGS. 12A and 12B) and hemagglutinin inhibition of influenza A/H1N1pdm09 (FIG. 13A) and influenza A/H3N2 (FIG. 13B). Data are represented as means±SEM. Pigs immunized with LHNVD-105 generated IgG antibodies that bound multiple strains of influenza virus 21 days post primary immunization (after a single dose) and had greater binding responses and neutralized multiple strains at d56 (after 1 boost). Pigs receiving low vaccine doses (1 and 5 μg) generated binding and neutralizing antibodies to influenza viruses equal to or higher than the high dose groups (50 and 100 μg).

### Example 4 Monoclonal Antibodies (mAbs) to Prevent or Treat Influenza Infections

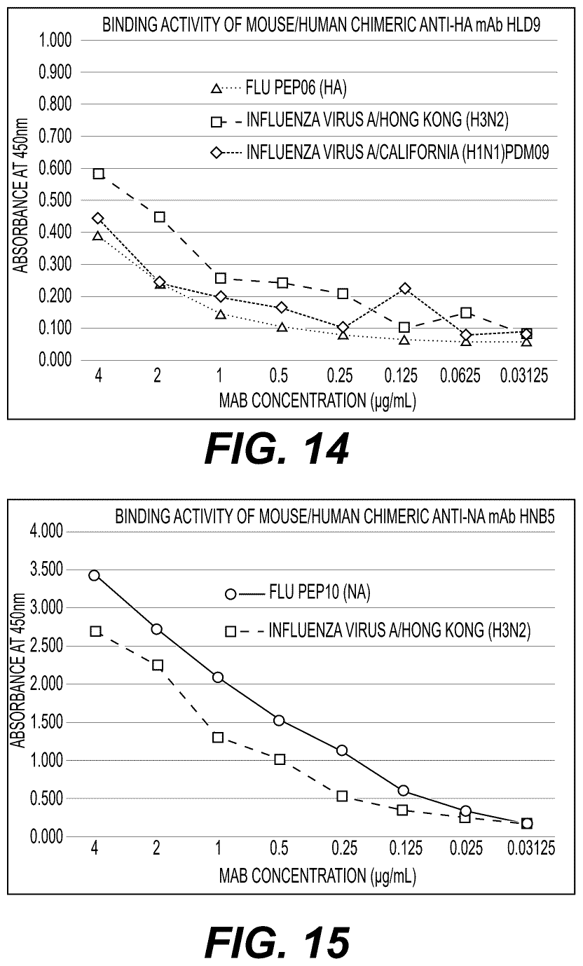

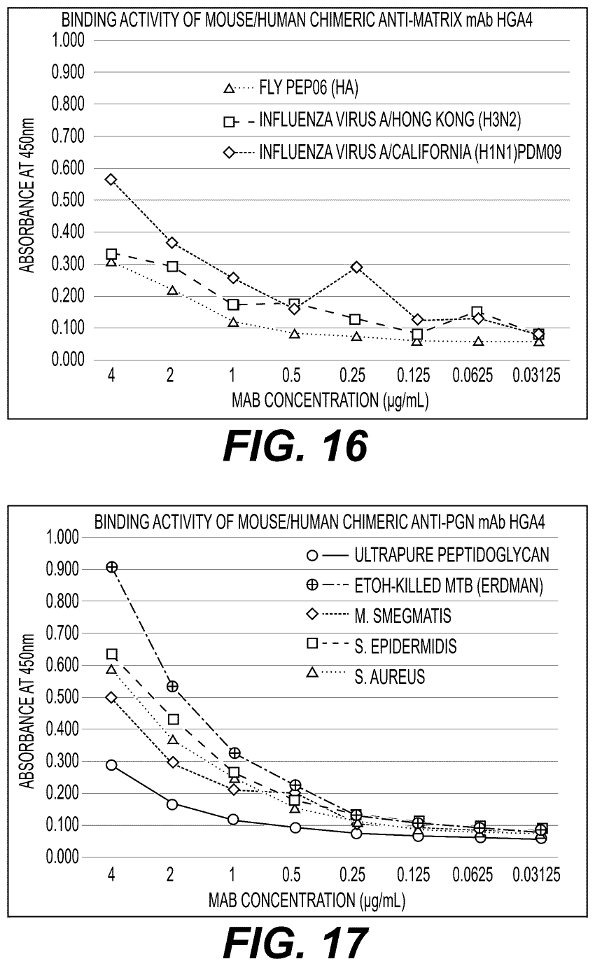

Broadly reactive human, humanized, or chimeric mAbs directed against composite peptides derived from hemagglutinin (HA), neuraminidase (NA), and matrix (M1/M2/M2e) viral surface proteins provide targeted therapies and mAb cocktails to multiple key influenza epitopes and broad therapeutic strategies against influenza. An anti-HA human/chimeric mAb hLD9 demonstrated good binding activity to Flu Pep06 (HA) and influenza A Group 1 (H1N1pdm09) and Group 2 (H3N2) viruses (see FIG. 14). The anti-NA human/chimeric mAb hNB5 demonstrated good binding activity to Flu Pep10 (NA) and influenza A Group 2 (H3N2) virus (see FIG. 15). The anti-Matrix human/chimeric mAb hGA4 demonstrated good binding activity of Flu Pep5906 (Matrix-M1, M2, M2e) and influenza A Group 1 (H1N1pdm09) and Group 2 (H3N2) viruses (see FIG. 16). One such mAb cocktail for prevention or treatment of influenza combines anti-HA and anti-NA and anti-Matrix (M1/M2/M2e) mAbs to enhance neutralization of influenza virus. In addition, these humanized or chimeric mAbs have mutations in the Fc region and an extended half-life and offer an extended anti-influenza prevention or treatment option.

### Example 5 Antimicrobial Resistance (AMR): Monoclonal Antibodies to Prevent or Treat Gram Positive Infections and Sepsis

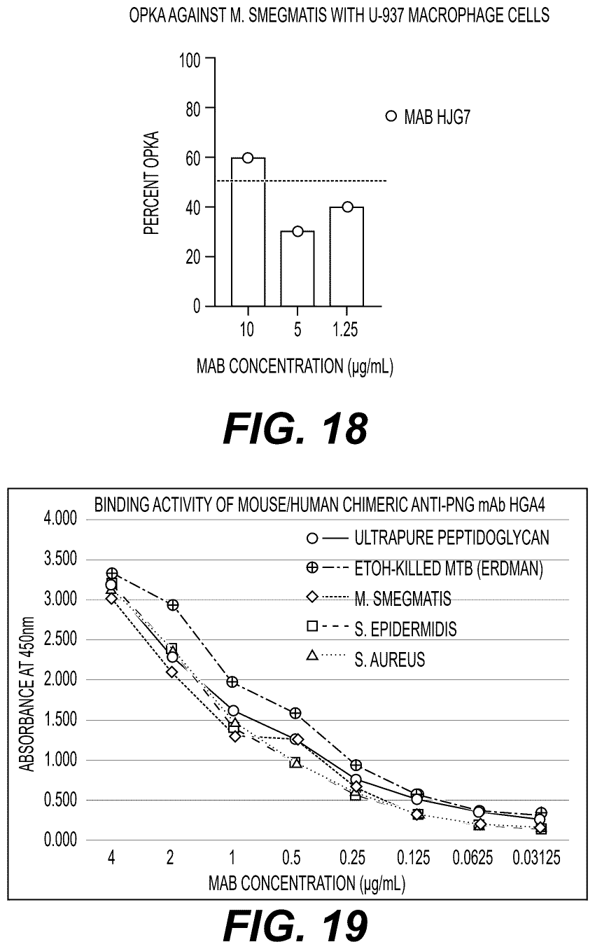

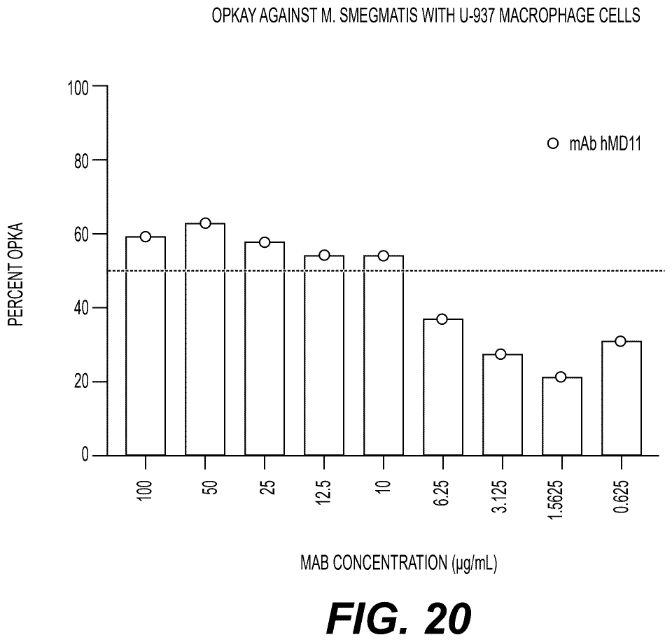

Antimicrobial resistance (AMR) has become a significant global threat. Monoclonal antibodies (mAbs) to highly conserved microbial epitopes that are immune enhancing (IE) or inhibit toxins (IT) could provide important strategies to treat AMR organisms. Human, or humanized mAbs provide targeted therapies and mAb cocktails to multiple key microbial epitopes and broad antimicrobial therapy. Anti-Peptidoglycan (PGN) humanized/chimeric mAb hJG7 (IgG1) showed good binding activity to ultrapure PGN from Staphylococcus aureus (SA), ethanol-killed Mycobacterium tuberculosis (MTB), Mycobacterium smegmatis (SMEG), Staphylococcus epidermidis (SE), and SA (see FIG. 17). Additionally, mAb hJG7 demonstrated significant opsonic activity against SMEG, using a macrophage cell line U-937 (see FIG. 18). Anti-PGN humanized/chimeric mAb hMD11 (IgG1) showed good binding activity to ultrapure PGN from Staphylococcus aureus (SA), ethanol-killed Mycobacterium tuberculosis (MTB), Mycobacterium smegmatis (SMEG), Staphylococcus epidermidis (SE), and SA (see FIG. 19). Additionally, mAb hMD11 demonstrated significant opsonic activity against SMEG, using a macrophage cell line U-937 (see FIG. 30). One such mAb cocktail for prevention or treatment of staphylococcal, or gram-positive infections combines anti-PGN and anti-LTA mAbs to both enhance opsonophagocytic killing (OPKA) of the organism and promote toxin inhibition and clearance. In addition, these humanized or chimeric mAbs may have a mutation in the Fc region to have an extended half-life. For example, serum half-life of IgG Abs is regulated by the neonatal Fc receptor (FcRn) such that binding to FcRn in endosomes, Abs are salvaged from lysosomal degradation and recycled. A typical antibody half-life is about 21 days. By substituting amino acid residues within the CH₂—CH₃ interface of Fc fragments half-life can be increased 2-3 fold and more (e.g., see U.S. Pat. No. 9,505,826 and US Patent Application Publication No. 2019/0016828, each of which is incorporated by reference). Adding an anti-LPS (endotoxin) mAb, gram negative bacteria will also be targeted in a broad-spectrum anti-sepsis mAb cocktail and adding the MTB heat shock protein mAb to the anti-PGN mAb and provide an extended half-life anti-TB prevention or treatment.

### Example 6 Exemplary Sequences

Conserved sequences from various bacterial, viral, and parasitic genomes, and sequences containing multiple different epitopes are provided below. These sequences contain epitopes and/or mimotopes (listed next to the sequence), that are recognized by mammalian immune systems. Epitopes are not sequences themselves, but structures recognized by immune systems components such as lymphocytes (T and B cells), macrophages, dendritic cells, neutrophils, eosinophils, basophils, mast cells, and natural killer (NK) cells. As such, there is variability to sequence of amino acids as the structure determines the response, not the specific sequence. Individual amino acids of a sequence may be altered without altering the epitope structure and, in fact, individual amino acids do change from one organism to another without altering the fact that the sequence contains a specific epitope. Additional bacterial, viral, and other epitopes and antibodies and their corresponding paratopes are disclosed and discussed in U.S. Pat. Nos. 12,220,387; 12,076,390; 12,043,647; 12,023,384; 11,872,273; 11,851,501; 11,640,847; 11,560,409; 11,439,702; 10,815,294; 10,774,134; and 8,470,340, each of which is specifically incorporated by reference.

Sequences of Mycobacteria
SEQ ID NO 1:
SEFAYGSFVRTVSLPVGADE (heat shock protein “HSP”
epitope)
SEQ ID NO 2:
SEFAYGSFMRSVTLPPGADE (Acr epitope M. smegmatis)
SEQ ID NO 3:
SEFSYGSFVRTVSLPGGADE (Acr epitope M. kansasii)
SEQ ID NO 4:
SEFSYGSFVRTVSLPAGADE (Acr epitope M. mantenii)
SEQ ID NO 5:
SEFSYGSFVRTVTLPTDADE (Acr epitope M. fortuitum)
SEQ ID NO 6:
SEFSYGSFARTVSLPAGANE (Acr epitope M. xenopi)
SEQ ID NO 7:
WRMYFSHRHAHLRSP (LTA epitope)
SEQ ID NO 8:
WHWRHRIPLQLAAGR (LTA epitope)
SEQ ID NO 9:
HSFKWLDSPRLR (Lipoarabinomannan “LAM” mimotopes)
SEQ ID NO 10:
ISLTEWSMWYRH (LAM mimotopes)
SEQ ID NO 11:
ISLTEWSMWYRHHSFKWLDSPRLR (LAM and LAM mimotopes)
SEQ ID NO 12:
SEFAYGSFVRTVSLPVGADEHSFKWLDSPRLR (Acr epitope)
SEQ ID NO 13:
AEKAGGGGGAEKA (peptidoglycan “PGN” epitope)
SEQ ID NO 14:
AEKAEKAGGGGGAEKAEKA (PGN epitope)
SEQ ID NO 15:
AEKAGGGGGAEKAQYIKANSKFIGITE (PGN epitope)
SEQ ID NO 16:
AEKA (PGN epitope)
SEQ ID NO 17:
AEKAGGGGG (PGN epitope)
Sequences of Influenza Virus (Flu)
SEQ ID NO 18:
DWSGYSGSFVQHPELTGLD (epitope of influenza N1
and N5)
SEQ ID NO 19:
KSCINRCFYVELIRGR (epitope of N3)
SEQ ID NO 20:
FVIREPFISCSHLEC (conserved epitope of influenza
N1 and N5)
SEQ ID NO 21:
GNFIAP (hemagglutinin “HA” epitope)
SEQ ID NO 22:
GNLIAP (HA epitope)
SEQ ID NO 23:
ETPIRNE (conserved epitope of N1)
SEQ ID NO 24:
HYEECSCY (neuraminidase “NA” epitope)
SEQ ID NO 25:
IWGIHHP (HA epitope)
SEQ ID NO 26:
IWGVHHP (HA epitope)
SEQ ID NO 27:
LLTEVETPIR (matrix “M1/M2e” epitope)
SEQ ID NO 28:
LLTEVETPIRNESLLTEVETPIRNEWG (M2e epitope)
SEQ ID NO 29:
LLTEVETPIRNEW (Matrix “M2e” epitope)
SEQ ID NO 30:
LLTEVETPIRNEWG (M2e epitope)
SEQ ID NO 31:
LTEVETPIRNE (M2e epitope)
SEQ ID NO 32:
LTEVETPIRNEW (M2e epitope)
SEQ ID NO 33:
LTEVETPIRNEWG (M2e epitope)
SEQ ID NO 34:
MSLLTEVET (M2e epitope)
SEQ ID NO 35:
MSLLTEVETP (M2e epitope)
SEQ ID NO 36:
MSLLTEVETPI (M2e epitope)
SEQ ID NO 37:
MSLLTEVETPIR (M2e epitope)
SEQ ID NO 38:
MSLLTEVETPIRN (M2e epitope)
SEQ ID NO 39:
MSLLTEVETPIRNE (M2e epitopes)
SEQ ID NO 40:
MSLLTEVETPIRNETPIRNE (M2e epitope)
SEQ ID NO 41:
MSLLTEVETPIRNEW (M2e epitope)
SEQ ID NO 42:
MSLLTEVETPIRNEWG (M2e epitope)
SEQ ID NO 43:
MSLLTEVETPIRNEWGCRCNDSSD (M2e epitope)
SEQ ID NO 44:
SLLTEVET (M2e epitope)
SEQ ID NO 45:
SLLTEVETPIRNE (M2e epitope)
SEQ ID NO 46:
SLLTEVETPIRNEW (M2e epitope)
SEQ ID NO 47:
SLLTEVETPIRNEWG (M2e epitope)
SEQ ID NO 48:
SLLTEVETPIRNEWGTPIRNE (M2e epitope)
SEQ ID NO 49:
SLLTEVETPIRNEWGTPIRNETPIRNE (M2e epitope)
SEQ ID NO 50:
SLLTEVETPIRNEWGTPIRNETPIRNETPIRNE (M2e epitopes)
SEQ ID NO 51:
SLLTEVETPIRNEWGLLTEVETPIRQYIKANSKFIGITE (M2e
epitope)
SEQ ID NO 52:
TEVETPIRNE (M2e epitope)
SEQ ID NO 53:
WGIHHP (HA conserved epitope)
SEQ ID NO 54:
WGVHHP (HA conserved epitope)
Sequences of Coronavirus (Cor)
SEQ ID NO 55:
YPKCDRA (RNA Polymerase “Pol” epitope)
SEQ ID NO 56:
WDYPKCDRA (RNA Polymerase conserved epitope)
SEQ ID NO 57:
SLDQINVTFLDLEYEMKKLEESY (spike protein conserved
epitope)
SEQ ID NO 58:
KWPWYIWLGFIAGL (spike protein conserved epitope)
SEQ ID NO 59:
ENQKLIAN (spike protein “SP” conserved epitope)
SEQ ID NO 60:
DLWSYNAELLV (stem peptide epitope)
SEQ ID NO 61:
DIWTYNAELLV (stem peptide epitope)
SEQ ID NO 62:
SLDQINVTFLDLEYEMKKLEESY (spike protein “SP”
epitope)
SEQ ID NO 63:
WDYPKCDRASLDQINVTFLDLEYEMKKLEESY (Pol + SP
epitopes)
SEQ ID NO 64:
ARDLICAQ (conserved SP attachment epitope for
Cor, MERS, SARS)
SEQ ID NO 65:
YFPLQSYGFQPTNGVGYQPYR (epitope)
Sequences of Staphylococcus and other bacteria
SEQ ID NO 66:
WRMYFSHRHAHLRSP (LTA Epitope)
SEQ ID NO 67:
WHWRHRIPLQLAAGR (LTA Epitope)
SEQ ID NO 68:
QEINSSY (lipopolysaccharide “LPS” epitope)
SEQ ID NO 69:
APPHALS (LPS epitope)
SEQ ID NO 70:
VVPTPPY (LPS epitope)
SEQ ID NO 71:
SMPNPMV (LPS epitope)
SEQ ID NO 72:
GLQQVLL (LPS epitope)
SEQ ID NO 73:
ELAPDSP (LPS epitope)
SEQ ID NO 74:
STLNYMYXAHPF (E. coli LPS epitope)
SEQ ID NO 75:
ISLSNIVDSQTP (S. typhi and E. coli LPS epitope)
SEQ ID NO 76:
GFSVITGAAMFE (Lipid A epitope of S. urbana and
E. coli)
Sequences of HIV (Human Immunodeficiency Virus)
SEQ ID NO 77:
RKSIHLGPGRAFY (HIV1 epitope)
SEQ ID NO 78:
KKGIAIGPGRTLY (HIV2 epitope)
SEQ ID NO 79:
RKSIRIGPGQAFY (HIV3 epitope)
SEQ ID NO 80:
RKRIRVGPGQTVY (HIV4 epitope)
Sequences of Plasmodium falciparum (Malaria)
SEQ ID NO 81:
NPDPNANPNVDPNANGGGC (malaria epitope)
Composite Sequences
SEQ ID NO 82:
GNLFIAP (composite Flu/HA epitope)
SEQ ID NO 83:
GNLIFAP (composite Flu/HA epitope)
SEQ ID NO 84:
IWGVIHHP (composite Flu/HA epitope)
SEQ ID NO 85:
IWGIVHHP (composite Flu/HA epitope)
SEQ ID NO 86:
WGVIHHP (composite Flu/HA epitope)
SEQ ID NO 87:
WGIVHHP (composite Flu/HA epitope)
SEQ ID NO 88:
YIWGVIHHP (composite Flu/HA epitope)
SEQ ID NO 89:
YIWGIVHHP (composite Flu/HA epitope)
Mixed Epitopes of Different Conserved Sequences
SEQ ID NO 90:
GNLFIAPWGVIHHPHYEECSCY (composite Flu HA/NA
epitopes)
SEQ ID NO 91:
WGVIHHPGNLFIAPHYEECSCY (composite Flu HA/NA and M
epitopes)
SEQ ID NO 92:
SEFAYGSFVRTVSLPVGADEGNLFIAPWGVIHHPHYEECSCY
(composite of TB HSP/Flu HA, HA and NA epitopes)
SEQ ID NO 93:
GNLFIAPWGVIHHPHYEECSCYSEFAYGSFVRTVSLPVGADE
(composite of Flu HA, HA and NA with MTB HSP
epitopes)
SEQ ID NO 94:
HYEECSCYSEFAYGSFVRTVSLPVGADE (composite of Flu
NA/MTB HSP epitopes)
SEQ ID NO 95:
SLLTEVETPIRNEWGLLTEVETPIR ()
SEQ ID NO 96:
HYEECSCYWDYPKCDRAVETPIRNE (Cor and Influenza
epitopes)
SEQ ID NO 97:
ENQKLIANTEVETPIRNEHYEECSCY (Cor SP/influenza M
and NA epitopes)
SEQ ID NO 98:
HYEECSCYHSFKWLDSPRLR (Influenza NA epitope/LAM
MTB mimotope)
SEQ ID NO 99:
NPDPNANPNVDPNANGGGCRKSIHLGPGRAFY (malaria/HIV1
epitope)
SEQ ID NO 100:
STLNYMYXAHPFWRMYFSHRHAHLRSPGGGGGAEKA
(LPS/LTA/PGN epitopes)
SEQ ID NO 101:
NMMRFTSQPPNNGGGGGAEKA (LOS H. Flu/PGN epitopes)
SEQ ID NO 102:
NPDPNANPNVDPNANGGGCHSFKWLDSPRLRRKSIRIGPGQAFY
(Malaria/TB LAM/HIV3 epitopes)
SEQ ID NO 103:
AEKAGGGGGHSFKWLDSPRLRSEFAYGSFVRTVSLPVGADE
(PGN/TB LAM and TB HSP epitopes)

This disclosure includes combinations of the epitopes including those listed above. Exemplary combinations include, but are not limited to, single peptides containing any one or more od SEQ ID NOs: 1-6 plus any one or more of SEQ ID NOs: 7 and 8, or 9-11, or 12-17; any one or more of SEQ ID NOs: 18-20 plus any one or more of SEQ ID NOs: 21-59, especially any combination of HA, NA and/or M1/M2e epitopes. Exemplary combinations further include, but are not limited to, single peptides containing any one or more od SEQ ID NOs: 55, 56, and/or 63 plus any one or more of SEQ ID NOs: 57-59, and/or 62, especially any combination of Pol, SP, and/or stem epitopes of Coronavirus. Exemplary combinations further include, but are not limited to, single peptides containing any one or more of SEQ ID NOs: 66 and/or 67 plus any one or more of SEQ ID NOs: 68-76, especially any combination of an epitope of Mycobacteria, and/or an epitope of Influenza, and/or an epitope of Staphylococcus, and/or an epitope of E coli, and/or an epitope of Coronavirus, and/or an epitope of another microorganism. Exemplary combinations further include, but are not limited to, single peptides containing any one or more of SEQ ID NOs: 82-89 plus any other epitope disclosed herein and/or another epitope of a microorganism.

Other embodiments and uses of the invention will be apparent to those skilled in the art from consideration of the specification and practice of the invention disclosed herein. All references cited herein, including all publications and U.S. and foreign patents and patent applications, are specifically and entirely incorporated by reference including U.S. Patent Publication No. 20240091331 entitled “Vaccines and Antibodies for the Treatment and Prevention of Microbial Infections”, published Mar. 21, 2024, U.S. Patent Publication No. 20240123055 entitled “Vaccines and Antibodies for the Treatment and Prevention of Microbial Infections”, published Apr. 18, 2024, U.S. Patent Publication No. 20210246174A1 entitled “Immunogenic Compositions to Treat and Prevent Microbial Infections”, published Aug. 12, 2021, U.S. Pat. No. 9,821,047 entitled “Enhancing Immunity to Tuberculosis,” which issued Nov. 21, 2017, U.S. Pat. No. 9,598,462 entitled “Composite Antigenic Sequences and Vaccines” which issued Mar. 21, 2017, U.S. Pat. No. 10,004,799 entitled “Composite Antigenic Sequences and Vaccines” which issued Jun. 26, 2018, U.S. Pat. No. 8,652,782 entitled “Compositions and Method for Detecting, Identifying and Quantitating Mycobacterial-Specific Nucleic Acid,” which issued Feb. 18, 2014, U.S. Pat. No. 9,481,912 entitled “Compositions and Method for Detecting, Identifying and Quantitating Mycobacterial-Specific Nucleic Acid,” which issued Nov. 1, 2016, U.S. Pat. No. 8,821,885 entitled “Immunogenic Compositions and Methods,” which issued Sep. 2, 2014, and all corresponding U.S. Provisional and continuation applications relating to any of the foregoing patents. The term comprising, wherever used, is intended to include the terms consisting of, and consisting essentially of. Furthermore, the terms comprising, including, containing and the like are not intended to be limiting. It is intended that the specification and examples be considered exemplary only with the true scope and spirit of the invention indicated by the following claims.

1. A composition for the prevention or treatment of a microbial infection comprised of an amount of contiguous peptide sequence of multiple viral, bacterial and/or parasitic epitopes that, upon administration to a human, a mammal and/or an animal, generates an immune response to a viral, bacterial and/or parasitic pathogen from which the epitopes are derived, wherein the amount comprises from about 0.01 μg to about 10 μg per single dose to a subject.

2. The composition of claim 1, wherein the amount is from about 0.01 μg to about 0.1 μg per dose.

3. The composition of claim 1, wherein the amount is from about 0.1 μg to about 1 μg per dose.

4. The composition of claim 1, wherein the amount is from about 1 μg to about 2 μg per dose.

5. The composition of claim 1, wherein the amount is from about 2 μg to about 3 μg per dose.

6. The composition of claim 1, wherein the amount is from about 3 μg to about 4 μg per dose.

7. The composition of claim 1, wherein the amount is from about 4 μg to about 5 μg per dose.

8. The composition of claim 1, wherein the contiguous peptide sequence comprises an epitope of a SARS-COV-2 virus.

9. The composition of claim 8, wherein the SARS-COV-2 virus epitope is an epitope of a spike protein.

10. The composition of claim 1, wherein the contiguous peptide sequence comprises a epitope of an Mycobacterial cell surface.

11. The composition of claim 10, wherein the Mycobacterial cell surface epitope is an epitope of a lipopolysaccharide, a peptidoglycan, a lipoarabinomannan, a lipotechoic acid, or a combination thereof.

12. The composition of claim 1, wherein all epitopes along the peptide sequence are discontinuous epitopes.

13. The composition of claim 1, wherein all epitopes along the peptide sequence are continuous epitopes.

14. The composition of claim 1, wherein at least one epitope comprises an epitope of an influenza virus.

15. The composition of claim 14, wherein the influenza epitope comprises an epitope of HA protein, NA protein, M1 protein, M2 protein, M2e protein, or a combination thereof.

16. The composition of claim 1, wherein at least one epitope is repeated along the peptide sequence.

17. The composition of claim 1, wherein the multiple epitopes comprise a repeated sequence of the collected epitopes of Influenza virus M1 protein, M2 protein, and M2e protein.

18. The composition of claim 17, wherein the multiple epitopes are epitopes of HA and NA proteins, and a T cell stimulating epitope.

19. The composition of claim 1, further comprising T cell stimulating epitope obtained or derived from tetanus toxin, tetanus toxin heavy chain proteins, diphtheria toxoid, CRM, recombinant CRM, tetanus toxoid, Pseudomonas exoprotein A, Pseudomonas aeruginosa toxoid, Bordetella pertussis toxoid, Clostridium perfringens toxoid, Escherichia coli heat-labile toxin B subunit, Neisseria meningitidis outer membrane complex, Hemophilus influenzae protein D, Flagellin Fli C, Horseshoe crab Haemocyanin, and/or a fragment, derivative, or modification thereof.

20. The composition of claim 1, wherein the T cell stimulating epitope is at an N-terminus of, at a C-terminus of, or internal to the peptide.

21. The composition of claim 1, which comprises multiple influenza virus epitopes and multiple T cell stimulating epitopes.

22. The composition of claim 1, further comprising an adjuvant.

23. The composition of claim 22, wherein the adjuvant comprises Freund's adjuvant, ALFQ, ALFQA, ALFA, AS01, AS01b, a liposome adjuvant, saponin, lipid A, squalene, and/or modifications, emulsions, nanoemulsions, derivatives and combinations thereof.

24. The composition of claim 1, which treats or prevents a viral, a bacterial, and/or a parasitic infection.

25. The composition of claim 24, wherein the viral infection comprises a coronavirus infection, an HIV infection, an influenza A infection, a Corona virus infection, or an influenza B infection.

26. The composition of claim 24, wherein the bacterial infection comprises infection of a gram-positive microorganism, infection of a gram negative microorganism, a Mycobacterial infection, an MTB infection, or a Staphylococcus infection.

27. The composition of claim 24, wherein the parasitic infection comprises a malaria infection.

28. A method to treat or prevent an infection by a pathogen by administering the immunogenic composition of claim 1 to a collection of animals suspected of being or determined to be infected with the pathogen.

29. The method of claim 28, wherein the composition produces a systemic and/or mucosal immune response against the pathogen by the animal.

30. The method of claim 28, wherein administration is to a water or food supply or as an aerosol.

31. The method of claim 28, wherein administration is oral, sub-cutaneous, intra-muscular, intradermal, or intra-nasal.

32. A method to treat an infection by a pathogen by administering the immunogenic composition of claim 1 to a human suspected of being or determined to be infected with the pathogen.

33. The method of claim 32, wherein the composition produces a systemic and/or mucosal immune response against the pathogen by the human.

34. The method of claim 32, wherein administration is oral, sub-cutaneous, intra-muscular, intradermal, or intra-nasal.

35. A composition for the prevention or treatment of a microbial infection comprised of an amount of a nucleic acid that encode a contiguous peptide sequence of multiple viral, bacterial and/or parasitic epitopes that, upon administration to a human, a mammal and/or an animal, generates an immune response to a viral, bacterial and/or parasitic pathogen from which the epitopes are derived, wherein the amount comprises from about 0.01 μg to about 10 μg per dose.

36. The composition of claim 35, wherein the nucleic acid comprises DNA or RNA.

37. A container comprised of the composition of claim 1 in an amount for administration to a single subject.

38. The container of claim 37, which is comprised of glass or plastic.

39. The container of claim 37, which is a syringe.

40. A container comprised of the composition of claim 1 in an amount for administration to a multiple subjects.

41. The container of claim 40, which is comprised of glass or plastic.
